(19) United States
(12) Patent Application Publication
(10) Pub. No.: US 2008/0031931 A1
(43) Pub. Date: Feb. 7, 2008

(54) ORGANOTELLURIUM AND SELENIUM-BASED ANTIMICROBIAL FORMULATIONS AND ARTICLES

(75) Inventor: Valerie E. Gunn, Cape Neddick, ME (US)

Correspondence Address: WILMERHALE/BOSTON, 60 STATE STREET, BOSTON, MA, 02109, US

(73) Assignee: Andover Healthcare, Inc., Salisbury, MA (US)

(21) Appl. No.: 11/688,230

(22) Filed: Mar. 19, 2007

Related U.S. Application Data
(60) Provisional application No. 60/783,234, filed on Mar. 17, 2006.

(51) Int. Cl. A61K 9/70; A61K 31/095; A61K 31/19; A61K 31/205; A61P 31/00; A61K 31/755; A61K 33/04

(52) U.S. Cl. 424/448; 424/449; 424/702; 424/78.07; 514/554; 514/557; 514/706

(57) ABSTRACT

The invention provides compositions and methods for treating or preventing the spread of infectious disease through topical or cutaneous contact with a formulation or article containing an organotellurium compound, or an inorganic or organic selenium compound, or formulation thereof, capable of generating superoxide radicals in the presence of an agent of infectious disease such as bacteria, viruses, fungi and protozoa.

### CROSS-REFERENCE TO RELATED APPLICATIONS

This application claims the benefit under 35 U.S.C. §119(e) of U.S. Provisional Patent Application No. 60/783,234, filed on Mar. 17, 2006, entitled Selenium-Based Antimicrobial Formulations and Articles, which is incorporated herein by reference in its entirety.

### 1. BACKGROUND OF THE INVENTION

Humans and other animals are in a constant immune-system battle with agents of infectious disease, including bacteria and viruses, as well as pathogenic fungi and protozoa. These agents of infectious disease reside in the environment, and in the flora of the skin. A particular problem for healthcare professionals dealing with these infectious agents has been the development of antibiotic resistant bacteria, which are refractory to many of the antibiotic agents that initially promised to provide a reliable cure. Indeed, the Center for Disease Control (CDC) has recently made the issues of combating antimicrobial resistance and preventing emerging infectious diseases two of its top priorities (see ““Federal Register Notice on Draft Public Health Action Plan to Combat Antimicrobial Resistance” (2000) JAMA 284:434; (2000) MMWR 49:603; and “Preventing Emerging Infectious Diseases” published by the National Center for Infectious Diseases, Centers for Disease Control and Prevention, Atlanta, Ga.).

A particularly critical problem for the healthcare industry has been the development and spread of infections, specifically those caused by Staphylococcus aureus, within the hospital environment. Medical devices, such as intravascular catheters provide a method for delivering fluids, medications, and nutrients to patients, however their use is also frequently associated with hospital-spread infections. Indeed, approximately 50% of hospital patients require intravenous access, and about 1-10% of catheters used eventually become contaminated. The most common consequence of such contamination is phlebitis (venous inflammation), and the most serious consequence of contamination is sepsis (systemic toxic condition resulting from the body-wide spread of bacteria and/or their products through the blood from the focus of infection).

Adhesive tapes used in conjunction with catheters and other medical devices are uniquely vulnerable to facilitating the spread of such infections in hospitals. This is because they are generally not washed or sterilized once they have been unpackaged, and, further, because a single roll of tape is generally used by several clinicians and on many different patients, and thereby becomes exposed to many different individuals. Furthermore, such adhesive tapes are frequently handled using ungloved hands and applied in close contact to the intravascular insertion site for extended periods of time. Indeed, one study found surprisingly high levels of infectious bacteria, including Staphylococcus aureus, on the outer layer of rolls of medical tape (3M Transpore™) that were in use throughout a hospital in Toronto (see Redelmeier and Livesley (1999) J. Gen. Int. Med. 14: 373-5).

As a result of widespread public concern with such infectious bacteria, antimicrobial materials such as fabrics, fibers, polymers and even children's toys have become increasingly popular. Indeed, the domestic and international market for antimicrobial fabrics has grown significantly as a result of public awareness of these potential threats (see, Center for Disease Control and Prevention, Infection Control and Biosafety, Medical Data International. Report #RP-701530, 1992; and A. J. Rigby, et al., Fiber Horizons, December 1993, 42-460). Antimicrobial clothing can be used in medicine as well as other institutional uses for such applications such as masks, patient drapes, bandages, wipers and cover cloths. Although the demand for such antimicrobial articles is high, relatively few types of such articles are available, and not all of those available are both effective against a broad spectrum of bacteria and capable of sustained antimicrobial activity without being released into the environment or gradually chemically inactivated.

Research and development of durable functional fibers has advanced in recent years, including new methods of incorporating antibiotics as bactericidal agents directly into the polymers. The chemical and medical literature describes many compounds that have antimicrobial activity (i.e., are capable of destroying or suppressing the growth or reproduction of microorganisms, such as bacteria). For example, a number of traditional antimicrobials are described in Antibiotics, Chemotherapeutics, and Antibacterial Agents for Disease Control (M. Grayson, editor, 1982), and E. Gale et al., The Molecular Basis of Antibiotic Action 2d edition (1981). Although the mechanism of action of these antimicrobials varies, they generally function by one or more of the following manners: inhibition of cell wall synthesis or repair; alteration of cell wall permeability; inhibition of protein synthesis; or inhibition of synthesis of nucleic acids (DNA or RNA). For example, beta-lactam antibacterials act through inhibiting the essential penicillin binding proteins (PBPs) in bacteria, which are responsible for cell wall synthesis. As another example, quinolones act, at least in part, by inhibiting synthesis of DNA, thus preventing the cell from replicating.

At least since the 1870s, silver has been recognized as an antibacterial agent, and has been particularly noted for its ability to resist the development of drug-resistance in target bacteria. In general, silver cations (Ag⁺) are thought to possess antimicrobial activity because they are highly reactive chemical structures that bind strongly to electron donor groups containing sulfur, oxygen, or nitrogen that are present in microbial targets. The biological target molecules generally contain all these components in the form of thio, amino, imidazole, carboxylate, and phosphate groups. Silver ions act by displacing other essential metal ions such as calcium or zinc. The direct binding of silver ions to bacterial DNA may also serve to inhibit a number of important transport processes, such as phosphate and succinate uptake, and can interact with cellular oxidation processes as well as the respiratory chain. The silver ion-induced antibacterial killing rate is directly proportional to silver ion concentrations, typically acting at multiple targets. Indeed, for silver ion-based antimicrobial articles and devices to be effective as antimicrobial vectors, the silver ions with which they are impregnated must be slowly released into the environment so that they are free to contact and inhibit the growth of destructive microbes in the environment. Accordingly, the antimicrobial activity of silver-coated and silver-impregnated articles and devices is dependent upon the controlled release rate of the unbound, free silver ions they carry, and the continued antimicrobial efficacy of such silver-based antimicrobials is necessarily limited by the supply of free silver ions they retain.

Therefore there is a need for sustainable and effective antimicrobial agents that both avoid the formation of resistant microbes and can be adapted for use in topical formulations, as well as for the coating or impregnation of topically-applied articles such as medical and sports tapes and bandages.

### 2. SUMMARY OF THE INVENTION

The invention is based, in part, upon the finding that organic tellurium compounds possess potent antimicrobial activities. In addition, the invention is based upon the further finding that organic and inorganic forms of selenium also possess similar antimicrobial properties. Accordingly, the invention provides compositions and methods utilizing organic tellurium compounds, as well as organic and inorganic forms of selenium, in antimicrobial applications. Particularly useful antimicrobial applications of the invention include incorporation into tapes and bandages, as well as other medical articles, to kill and/or inhibit the growth and/or spreading of pathogens, including bacteria and other organisms.

The inventors have found that organic forms of tellurium are particularly effective as antimicrobial agents, and further, that the organic portion of such organotellurium compounds facilitate their incorporation and subsequent bioavailability in formulations, including formulations for topical applications such as in adhesive or cohesive tapes or bandages. The inventors have also found that both organic and inorganic selenium compounds may be utilized in a similar fashion. While not wishing to limit the invention to any particular compound(s) or mechanism of action, the inventors have found that organic tellurium compounds are particularly potent antimicrobials for use in such formulations and act, at least in part, by generating antimicrobial superoxide molecules in the presence of reduced chemical moieties, such as sulfhydryl groups that are present on or within microbes, including bacteria and pathogenic fungi. The resulting superoxide (hyperoxide ion, O₂⁻) are generated in close proximity to the microbial organism and provide a short-lived, but highly reactive germicidal oxidation activity.

In general, the invention provides antimicrobial compositions incorporating organotellurium and organoselenium compounds, as well as methods of use of such antimicrobial compositions. In addition, the invention provides antimicrobial compositions incorporating inorganic selenium, and associated methods of their use. While not wishing to limit the invention to any particular compound(s), the invention provides that, in general, the antimicrobial potency of these compounds is generally in the order of;
[Organotellurium]>[Organoselenium]>[Inorganic Selenium]>>[Inorganic tellurium], or, in general chemical structural terms:
[R—Te—(Te)—R′]>[R—Se—(Se)—R′]>[ᵢSe]>>[ᵢTe],
where R—Te—(Te)—R′ represents the general chemical structure of the organotellurium compounds of the invention, Te being tellurium, R being any organic (i.e., carbon-based) chemical group, (Te) being an optional second atom of tellurium covalently bonded to the first, and R′ being an optional second organic (i.e., carbon based) chemical group that may be bonded directly to the first tellurium atom, or to the optional second tellurium atom (i.e., to (Te)). Organotellurium compounds generally include organic tellurides having the general structure R—Te or R—Te—R, as well as ditellurides having the general structure R—Te—Te—R′.
R—Se—(Se)—R′ represents the general chemical structure of the organoselenium compounds of the invention, Se being selenium, R being any organic (i.e., carbon-based) chemical group, (Se) being an optional second atom of selenium covalently bonded to the first, and R′ being an optional second organic (i.e., carbon based) chemical group that may be bonded directly to the first selenium atom, or to the optional second selenium atom (i.e., to (Se)). Organoselenium compounds generally include organic selenides having the general structure R—Se or R—Se—R, as well as diselenides having the general structure R—Se—Se—R′.
ᵢSe represents inorganic forms of selenium including selenates and selenites. Selenates are inorganic forms of Se in which selenium is the central atom(s) in an anion, such as M₂SeO₄, where M is a monovalent metal atom, while selenites are inorganic forms of selenium that are salts of selenious acid having the general structure M₂SeO₃. ᵢSe generally includes selenic compounds of Selenium(IV) or (4+), as well as Selenium(VI) or (6+). ᵢSe also generally includes selenious compounds of Selenium(II) or (2+), as well as Selenium (IV) or (4+).
ᵢTe represents inorganic forms of tellurium, including tellurites. Tellurites are inorganic forms of Te in which tellurium is the central atom in an anion, such as M₂TeO₃, where M is a monovalent metal atom, Tellurate(IV) (+4) (e.g., TeO₃⁻² ion). ᵢTe generally also includes telluric compounds of Tellurium(VI) or (6+). ᵢTe also generally includes tellurous compounds of Telenium(II) or (2+), as well as Tellurium (IV) or (4+).

The invention is further based, upon the finding that organotellurium compounds, as well as organic and inorganic selenium compounds that catalyze the formation of free radical superoxide ions in the presence of oxygen and a reducing agent such a reduced thiol group, provide antimicrobial activity when applied topically, and, particularly cutaneously, to a subject. While not wishing to be bound by a single theory of their mechanism of cutaneous antimicrobial action, such selenium-containing compounds appear to provide for catalytic superoxide-mediated damage to target microbes such as bacteria by generating short-lived but highly reactive superoxide (O₂⁻) ions in the presence of oxygen (O₂) and reduced thiol groups (SH— groups) present on the target microbe itself (e.g., from membrane proteins or other reducing sources present on or near the target microbe). Accordingly, the invention provides novel, topical, selenium-based methods, formulations and articles for the treatment or prevention of infectious, disease-causing agents.

In one aspect, the invention provides an antimicrobial article that includes a substrate and an organotellurium compound on at least one surface of the substrate. The organotellurium compound is present on the surface of the substrate in an amount sufficient to treat or prevent the growth or spread of an infectious agent through cutaneous contact of the antimicrobial article with a subject, such as a human subject. In certain embodiments, the article includes an organotellurium compound having the structure:
R—Te,
where Te is tellurium and R is any organic chemical group. In other embodiment, the antimicrobial article includes an organotellurium compound having the structure:
R—Te—Te—R′,
where Te are tellurium atoms, and R and R′ are each, independently, any organic chemical group. In still further embodiments, the organotellurium compound includes an R group that is a substituted or unsubstituted, saturated or unsaturated, alkyl group having from 1 to about 12 carbon atoms. In further embodiments, the alkyl group is substituted with one or more substituents selected from the group consisting of methyl, amino, halo (e.g., chloro), nitro, methoxy, hydroxy, carboxylate, vinyl, allyl, alkylsilane and combinations thereof. In other embodiments, the organotellurium compound includes an R group that is a substituted or unsubstituted, saturated or unsaturated, aryl group. In further embodiments, the aryl R group is phenyl, pyridinium, imidazole, oxazine, or naphthyl. In still further embodiments, the aryl group is substituted with one or more substituents such as methyl, amino, halo(chloro), nitro, methoxy, hydroxy, carboxylate, vinyl, allyl, alkylsilane or combinations thereof. In further embodiments, the organotellurium compound is an organotellurium carboxylic acid compound, or alkyl ester thereof. In still further embodiments, the organotellurium carboxylic acid compound, or alkyl ester thereof, is tellurocyanatoacetic acid, 3-tellurocyanatopropionic acid, 2-tellurocyanotopropionic acid, ditellurocyanatodiacetic acid, ditellurocyanatodipropionic acid, or mixtures thereof.

In yet other embodiments, the antimicrobial article includes, on at least one surface, an organotellurium compound such as tellurocyanatoethyl amine, 3-tellurocyanatopropyl amine, tellurocystamine dihydrochloride or 2-tellurocyanatoethyl methacrylate. In further embodiments, the antimicrobial article is coated, on at least one surface, with an organotellurium compound such as tellurodicarboxylic acid, organotellurium diamines, organotellurium monoamines (and amides thereof), organotellurium amines (e.g., cystamine), telluroamino acids (e.g., L-tellurocystine), organotellurium carboxylic acids and amides thereof), telluroacrylates and esters and amides thereof, telluromethacrylates and esters and amides thereof, tellurourethanes, telluroureas, and tellurofatty acids and esters and amides thereof), tellurochloroprene, tellurobromoprene, tellurostyrene, tellurobutadiene, telluroacrylonitrile, or mixtures thereof. In further embodiments, the organotellurium compound is a telluro adduct of oleic acid, or an alkyl ester thereof. In yet other embodiments, the organotellurium compound is a polymer, or mixture of polymers, selected from the group consisting of telluroamino acids, 2-substituted organotellurium oxazolines, 2-substituted organotellurium oxazines, telluroacrylate, telluromethacrylate, tellurourethane, tellurourea, tellurochloroprene, tellurobromoprene, tellurostyrene, tellurobutadiene, and telluroacrylonitrile. In particular embodiments, the organotellurium compound is 2,2′-tellurodiethanamine dihydrochloride or 2,2′-ditellurodiethanamine dihydrochloride.

In other embodiments, the antimicrobial article includes, on at least one surface, an organotellurium compound that is present at a concentration of at least about 3 μg/cm² of Te (about 23.5 nmole/cm² Te). In further embodiments, the organotellurium compound is present at no more than about 200 μg/cm² of Te (about 1.57 μmole/cm² Te). In still other embodiments, the organotellurium compound is present at a concentration ranging from about 3 μg/cm² of Te (about 23.5 nmole/cm² Te) to about 100 μg/cm² of Te (about 784 nmole/cm² Te). In further embodiments, the organotellurium compound is present at about 10 μg/cm² of Te (about 78.4 nmole/cm² Te) to about 20 μg/cm² of Te (about 157 nmole/cm² Te). In particular embodiments, the article comprises at least one surface having about 3 ug of elemental tellurium per square centimeter of surface area.

In certain particularly useful embodiments, the organotellurium compound is non-covalently associated with the article. In other embodiments, the organotellurium compound is covalently associated with the article. In further embodiments, the antimicrobial article includes an organotellurium compound that is formulated into a formulation that is applied to at least one surface of the antimicrobial article. In particular embodiments, the formulation is a pressure-sensitive adhesive. In other embodiments, the formulation is a cohesive agent. In particularly useful embodiments, at least about 50% of the organotellurium is bioavailable at the surface of the article. In further embodiments, at least about 70% of the organotellurium is bioavailable at the surface of the article. In certain embodiments the antimicrobial article is an antimicrobial tape, such as a medical tape or a sports tape. In other embodiments, the antimicrobial article is an antimicrobial bandage, such as a medical bandage or a sports bandage.

In another aspect the invention provides a method of treating or preventing an infectious disease spread through cutaneous contact of a subject with a medical or sports article by contacting the subject topically with a medical or sports article having, on at least one surface, an organic tellurium compound, or formulation thereof, capable of generating superoxide radicals in the presence of an infectious agent. By this aspect of the invention, the superoxide radicals generated by the organic tellurium compound inhibit or inactivate the agent of the infectious disease and thereby treat or prevent the infectious disease in the subject.

In still another aspect of the invention, the invention provides a method of treating or preventing the development or transmission of an infectious disease in a subject through the use of an adhesive or cohesive article applied cutaneously to the subject by first providing an adhesive or cohesive article having, on at least one surface, an organic tellurium compound, or formulation thereof, capable of generating superoxide radicals in the presence of an infectious agent at a surface of the article; and then applying the surface of the adhesive or cohesive article to the subject. By this aspect of the invention, the superoxide radicals generated by the organic tellurium compound at the applied surface of the article inhibit or inactivate the agent of the infectious disease and thereby treat or prevent the infectious disease in the subject.

In still another aspect, the invention provides a method of making an antimicrobial adhesive or cohesive formulation by, providing an adhesive or cohesive formulation; and adding to the adhesive or cohesive formulation an organic tellurium compound, or formulation thereof, capable of generating superoxide radicals in the presence of an infectious agent so as to make the antimicrobial adhesive or cohesive formulation.

In another aspect, the invention provides a method of making an antimicrobial adhesive or cohesive article by: providing an adhesive or cohesive formulation; adding to the adhesive or cohesive formulation an organic tellurium compound, or formulation thereof, capable of generating superoxide radicals in the presence of an infectious agent to produce an antimicrobial adhesive or cohesive formulation; and applying the antimicrobial adhesive or cohesive formulation to at least one surface of the article, so as to produce the antimicrobial adhesive or cohesive article.

In particular embodiments of the above four methods of the invention, the agent of infectious disease is a bacteria, such as Staphylococcus aureus (e.g., MRSa), Pseudomonas aeruginosa, or Acinetobacter baumanii. In further embodiments, the infectious agent is a fungus. In still other embodiments, the infectious disease is caused by a protozoa. In still other embodiments, the infectious disease is caused by a virus.

In further embodiments of the above four methods of the invention, the organic tellurium compound, or formulation thereof, does not include a thiol group or a thiol-containing compound. In particular embodiments, the organic tellurium compound, or formulation thereof, does not include glutathione.

In certain further embodiments of the above four methods of the invention, the subject is a mammal or a marsupial. In particular embodiments, the mammal is a mouse, a rat, a dog, a cat, a cow, a horse, a goat or a pig. In particularly useful embodiments, the subject is a human.

In still further embodiments of the above four methods of the invention, the organic tellurium compound is tellurocystine or telluromethionine. In other embodiments, the organotellurium compound is a carboxylic acid compound, or alkyl ester thereof, such as tellurocyanatoacetic acid, 3-tellurocyanatopropionic acid, 2-tellurocyanatopropionic acid, ditellurocyanatodiacetic acid, ditellurocyanatodipropionic acid, or mixtures thereof. In yet other embodiments, the organotellurium compound is tellurocyanatoethyl amine, 3-tellurocyanatopropylamine, tellurocystamine dihydrochloride or 2-(tellurocyanatoethyl methacrylate. In further embodiments, the organotellurium compound is tellurodicarboxylic acid, an organotellurium diamine, an organotellurium monoamine (or an amide thereof), an organotellurium amine (e.g., cystamine), a telluroamino acid (e.g., L-tellurocystine), an organotellurium carboxylic acids (or amide thereof), a telluroacrylate (or ester or amide thereof), a telluromethacrylates (or esters or amides thereof), a tellurourethane, a tellurourea, a tellurofatty acids (or esters or amide thereof), a tellurochloroprene, a tellurobromoprene, a tellurostyrene, a tellurobutadiene, a telluroacrylonitrile, or mixtures of any of these types of organotellurium compounds. In particular embodiments, the organotellurium compound is a telluro adduct of oleic acid, or an alkyl ester thereof. In other embodiments, the organotellurium compound is a polymer, or mixture of polymers, of an organotellurium compound(s) such as telluroamino acids, 2-substituted organotellurium oxazolines, 2-substituted organotellurium oxazines, telluroacrylate, telluromethacrylate, tellurourethane, tellurourea, tellurochloroprene, tellurobromoprene, tellurostyrene, tellurobutadiene, or telluroacrylonitrile.

In some embodiments of the above four methods of the invention, the organic tellurium compound, or formulation thereof, is non-covalently associated with the article. In other embodiments, the organic tellurium compound, or formulation thereof, is covalently associated with the article. In particular embodiments the article is an adhesive tape or bandage. In other embodiments, the article is a cohesive tape or bandage. In further embodiments, the surface of the article having the organic tellurium compound, or formulation thereof, further includes a pressure-sensitive adhesive composition. In other embodiments, the adhesive or cohesive formulation is a pressure-sensitive adhesive. In particular embodiments, the pressure-sensitive adhesive is an acrylic water-based or solvent-based pressure-sensitive adhesive. In certain embodiments, the pressure-sensitive adhesive is a hot-melt adhesive.

In particularly useful embodiments of the above four methods of the invention, at least about half of the elemental tellurium from the organic tellurium compound, or formulation thereof, exists in an active state that is capable of generating superoxide radicals on the surface of the antimicrobial adhesive or cohesive article. In particular embodiments, the article has at least one surface having at least about 2 ug of elemental tellurium per square centimeter of surface area. In further embodiments, the article has at least one surface having at least about 6 ug of elemental tellurium per square centimeter of surface area.

In another aspect, the invention provides an antimicrobial article having, on at least one surface, an effective amount of an organic tellurium compound, or formulation thereof, capable of generating superoxide radicals in the presence of an infectious agent compound. By this aspect, the organic tellurium compound is non-covalently associated with the article and an effective amount of the organic tellurium compound, or formulation thereof, is retained on the surface of the article when the article is in cutaneous contact with a subject.

In still another aspect, the invention provides an antimicrobial adhesive or cohesive article having, on at least one surface, an antimicrobial cohesive or adhesive formulation including an effective amount of an organic tellurium compound, or formulation thereof, capable of generating superoxide radicals in the presence of an infectious agent or reduced thiol compound. By this aspect of the invention, the organic tellurium compound is non-covalently associated with the article and an effective amount of the organic tellurium compound, or formulation thereof, is retained on the surface of the article when the article is in cutaneous contact with a subject.

In yet another aspect, the invention provides an antimicrobial adhesive or cohesive article product of a process including the steps of: providing an adhesive or cohesive formulation; adding to the adhesive or cohesive formulation an organic tellurium compound, or formulation thereof, capable of generating superoxide radicals in the presence of an infectious agent to produce an antimicrobial adhesive or cohesive formulation; and applying the antimicrobial adhesive or cohesive formulation to at least one surface of the article to produce the antimicrobial adhesive or cohesive article. By this aspect of the invention, the resulting antimicrobial adhesive or cohesive article carries, on at least one surface, an effective amount of the organic tellurium compound, or formulation thereof, that is capable of generating superoxide radicals in the presence of an infectious agent.

In still another aspect, the invention provides an antimicrobial adhesive or cohesive article having, on at least one surface, an antimicrobial cohesive or adhesive formulation that includes an effective amount of an organic tellurium compound, or formulation thereof, capable of generating superoxide radicals in the presence of an infectious agent or a reduced thiol compound. By this aspect of the invention, the organic tellurium compound is covalently associated with the article and an effective amount of the organic tellurium compound, or formulation thereof, is retained on the surface of the article when the article is in cutaneous contact with a subject.

In some embodiments of the above four aspects of the invention, the antimicrobial article includes an amount of organic tellurium compound, or formulation thereof that is retained on the surface of the article when the article is in cutaneous contact with the subject, is sufficient to inhibit or inactivate an agent of infectious disease. In particular embodiments, the agent of infectious disease is a bacteria, such as Staphylococcus aureus. (e.g., MRSa), Pseudomonas aeruginosa or Acinetobacter baumannii. In other embodiments, the agent of infectious disease is a fungus. In further embodiments, the agent of infectious disease is a protozoa. In still further embodiments, the agent of infectious disease is a virus.

In further embodiments of the above four aspects of the invention, the antimicrobial article having the organic tellurium compound, or formulation thereof, does not further include a thiol group or a thiol-containing compound. In particular embodiments, the antimicrobial article does not further include glutathione.

In certain embodiments of the above four aspects of the invention, the subject is a mammal (e.g. a human).

In still further embodiments of the above four aspects of the invention, the antimicrobial article includes an organic tellurium compound such as tellurocystine or telluromethionine. In other embodiments, the organic tellurium compound is an organotellurium carboxylic acid compound, or alkyl ester thereof, such as tellurocyanatoacetic acid, 3-tellurocyanatopropionic acid, 2-tellurocyanatopropionic acid, ditellurocyanatodiacetic acid, ditellurocyanatodipropionic acid, or mixtures of any of these organotellurium carboxylic acid compounds. In yet other embodiments, the organic tellurium compound is an organotellurium compound such as tellurocyanatoethyl amine, 3-tellurocyanatopropyl amine, tellurocystamine dihydrochloride or 2-tellurocyanatoyethyl methacrylate. In still other embodiments, the organic tellurium compound is a telluro adduct of oleic acid, or an alkyl ester thereof.

In particularly useful embodiments of the above four aspects of the invention, the antimicrobial article is an adhesive tape or bandage. In other embodiments, the antimicrobial article is a cohesive tape or bandage. In still further embodiments, the surface of the article having the organic tellurium compound, or formulation thereof, further carries a pressure-sensitive adhesive. In still other embodiments, the antimicrobial article includes a pressure-sensitive adhesive that is either an acrylic water-based or a solvent-based pressure-sensitive adhesive. In particularly useful embodiments, the antimicrobial article includes a pressure-sensitive adhesive that is a hot-melt adhesive.

In further useful embodiments of the above four aspects of the invention at least about half of the elemental tellurium from the organic tellurium compound, or formulation thereof, exists in an active state that is capable of generating superoxide radicals on the surface of the antimicrobial article. In particular embodiments, the antimicrobial article has at least one surface having at least about 2 ug of elemental tellurium per square centimeter of surface area. In still further useful embodiments, the antimicrobial article has at least one surface having about 6 ug of elemental tellurium per square centimeter of surface area.

In yet another aspect, the invention provides an antimicrobial article that includes a substrate and an inorganic or organic selenium compound. By this aspect of the invention, the inorganic or organic selenium compound is present in an amount sufficient to treat or prevent the growth or spread of an infectious agent through cutaneous contact of the antimicrobial article with a subject. In particular embodiments, the organoselenium compound has the structure:
R—Se,
where Se is selenium and R is any organic chemical group. In other embodiments, the organoselenium compound has the structure:
R—Se—Se—R′,
where Se are selenium atoms, and R and R′ are each, independently, any organic chemical group. In certain embodiments, the organoselenium compound includes an R group that is a substituted or unsubstituted, saturated or unsaturated, alkyl group having from 1 to about 12 carbon atoms. In particular embodiments, the alkyl group is substituted with one or more substituents selected from the group consisting of methyl, amino, halo(chloro), nitro, methoxy, hydroxy, carboxylate, vinyl, allyl, alkylsilane and combinations thereof. In still other embodiments, the organoselenium compound includes an R group that is a substituted or unsubstituted, saturated or unsaturated, aryl group. In certain embodiments, the aryl group is a phenyl, pyridinium, imidazole, oxazine, or naphthyl aryl group. In particular embodiments, the aryl group is substituted with one or more substituents such as methyl, amino, halo(chloro), nitro, methoxy, hydroxy, carboxylate, vinyl, allyl, alkylsilane or combinations thereof. In further embodiments, the organoselenium compound is an organoselenium carboxylic acid compound, or alkyl ester thereof. In particular embodiments, the organoselenium carboxylic acid compound, or alkyl ester thereof, is selenocyanatoacetic acid, 3-selenocyanatopropionic acid, 2-selenocyanotopropionic acid, diselenocyanatodiacetic acid, diselenocyanatodipropionic acid, or a mixture of any of these. In further embodiments, the organoselenium compound is selenocyanatoethyl amine, 3-selenocyanatopropyl amine, selenocystamine dihydrochloride or 2-(selenocyanatoethyl methacrylate. In still other embodiments, the organoselenium compound is a selenodicarboxylic acid, an organoselenium diamine, an organoselenium monoamine (or an amide thereof), an organoselenium amine (e.g., a cystamine), a selenoamino acid (e.g., L-selenocystine), an organoselenium carboxylic acid, or amide thereof, a selenoacrylate or ester or amide thereof, a selenomethacrylate or ester or amide thereof, a selenourethane, a selenourea, a selenofatty acid or esters or amides thereof, a selenochloroprene, a selenobromoprene, a selenostyrene, a selenobutadiene, a selenoacrylonitrile, or a mixture of any of these. In particular embodiments, the organoselenium compound is a seleno adduct of oleic acid, or an alkyl ester thereof. In further embodiments, the organoselenium compound is a polymer, or mixture of polymers, of selenoamino acids, 2-substituted organoselenium oxazolines, 2-substituted organoselenium oxazines, selenoacrylate, selenomethacrylate, selenourethane, selenourea, selenochloroprene, selenobromoprene, selenostyrene, selenobutadiene, or selenoacrylonitrile. In particularly useful embodiments, the organoselenium compound is 2,2′-selenodiethanamine dihydrochloride or 2,2′-diselenodiethanamine dihydrochloride.

In further embodiments, the antimicrobial article includes an organoselenium compound that is present at a concentration of at least about 3 μg/cm² of Se (about 38.0 nmole/cm² Se). In some embodiments, the organoselenium compound is present at no more than about 200 μg/cm² of Se (about 2.54 μmole/cm² Se). In still further embodiments, the organoselenium compound is present at a concentration in the range from about 3 μg/cm² of Se (about 38.0 nmole/cm² Se) to about 100 μg/cm² of Se (about 1,266 nmole/cm² Se). In particularly useful embodiments, the organoselenium compound is present in the range from about 10 μg/cm² of Se (about 126.6 nmole/cm² Se) to about 20 μg/cm² of Se (about 253 nmole/cm² Se). In certain embodiments, the antimicrobial article includes at least one surface having about 3 ug of elemental selenium per square centimeter of surface area.

In particularly useful embodiments, the organoselenium compound is non-covalently associated with the article. In certain embodiments, the organoselenium compound is formulated in a formulation that is applied to at least one surface of the antimicrobial article. In other embodiments, the antimicrobial article the formulation containing the organoselenium compound is a pressure-sensitive adhesive. In other embodiments, the formulation containing the organoselenium compound is a cohesive agent.

In further particularly useful embodiments, at least 50% of the organoselenium is bioavailable at the surface of the article. In certain embodiments, at least 70% of the organoselenium is bioavailable at the surface of the article. In some embodiments, the antimicrobial article is a tape. In other embodiments, the antimicrobial article is a bandage.

In further embodiments, the antimicrobial article includes an inorganic selenium compound that is a salt of selenite (SeO₃⁻²). In particular embodiments, the selenite is sodium selenite (Na₂SeO₃).

In yet another aspect, the invention provides a method of treating or preventing an infectious disease spread through cutaneous contact of a subject with a medical or sports article. The method involves contacting the subject topically with a medical or sports article having, on at least one surface, an inorganic or organic selenium compound, or formulation thereof, capable of generating superoxide radicals in the presence of an infectious agent. The superoxide radicals generated by the inorganic or organic selenium compound inhibit or inactivate an agent of the infectious disease and thereby treat or prevent the infectious disease in the subject.

In another aspect, the invention provides a method of treating or preventing the development or transmission of an infectious disease in a subject through the use of an adhesive or cohesive article applied cutaneously to the subject. The method involves providing an adhesive or cohesive article having, on at least one surface, an inorganic or organic selenium compound, or formulation thereof, capable of generating superoxide radicals in the presence of an infectious agent at a surface of the article. A surface of the adhesive or cohesive article is then applied to the subject. The superoxide radicals generated by the inorganic or organic selenium compound at the applied surface of the article inhibit or inactivate the agent of the infectious disease and thereby treat or prevent the infectious disease in the subject.

In still another aspect, the invention provides a method of making an antimicrobial adhesive or cohesive formulation. The method involves the steps of providing an adhesive or cohesive formulation, and adding to the adhesive or cohesive formulation an inorganic or organic selenium compound, or formulation thereof, that is capable of generating superoxide radicals in the presence of an infectious agent, so as to make the antimicrobial adhesive or cohesive formulation.

In yet another aspect, the invention provides a method of making an antimicrobial adhesive or cohesive article. The method involves the steps of providing an adhesive or cohesive formulation; adding an inorganic or organic selenium compound, or formulation thereof, capable of generating superoxide radicals in the presence of an infectious agent, to the adhesive or cohesive formulation to produce an antimicrobial adhesive or cohesive formulation. The resulting antimicrobial adhesive or cohesive formulation is then applied to at least one surface of the article, so as to create the antimicrobial adhesive or cohesive article.

In certain embodiments of these methods of the invention, the agent of infectious disease that is being treated or prevented is a bacteria. In particular embodiments the bacteria is Staphylococcus aureus, Pseudomonas aeruginosa, or Acinetobacter baumanii. In further embodiments of the invention, the agent of infectious disease is a fungus, a protozoa, or a virus.

In other embodiments, the inorganic or organic selenium compound, or formulation thereof, does not comprise a thiol group or a thiol-containing compound. In particular embodiments, the inorganic or organic selenium compound, or formulation thereof, does not comprise glutathione.

In further embodiments, the subject in which infectious disease is treated or prevented is a mammal or a marsupial. In particular embodiments, the mammal a mouse, a rat, a dog, a cat, a cow, a horse, a goat, a pig, a kangaroo or a yak. In other embodiments the subject is a human.

In certain particularly useful embodiments, the inorganic or organic selenium compound is selected selenite, selenate, selenocystine or selenomethionine. In other embodiments, the selenium compound is an organoselenium carboxylic acid compound, or alkyl ester thereof, such as selenocyanatoacetic acid, 3-selenocyanatopropionic acid, 2-selenocyanatopropionic acid, diselenocyanatodiacetic acid, diselenocyanatodipropionic acid, or mixtures thereof. In still other embodiments, the selenium compound is an organoselenium compound such as selenocyanatoethyl amine, 3-selenocyanatopropyl amine or selenocystamine dihydrochloride. In still other useful embodiments, the selenium compound is an organoselenium compound such as selenodicarboxylic acid, organoselenium diamines, organoselenium monoamines (and amides thereof), organoselenium amines (e.g., cystamine), selenoamino acids (e.g., L-selenocystine), organoselenium carboxylic acids (and amides thereof), selenourethanes, selenoureas, and selenofatty acids (and esters and amides thereof), selenochloroprene, selenobromoprene, selenostyrene, selenobutadiene, selenoacrylonitrile, or a mixture of one or more of these compounds. In still other embodiments, the selenium compound is a seleno adduct of a fatty acid, or an alkyl ester thereof. In further embodiments, the selenium compound is a polymer, or mixture of polymers, of an organoselenium compound or compound family such as selenoamino acids, 2-substituted organoselenium oxazolines, 2-substituted organoselenium oxazines, selenourethane, selenourea, selenochloroprene, selenobromoprene, selenostyrene, selenobutadiene, or selenoacrylonitrile.

In further particular embodiments, the inorganic or organic selenium compound, or formulation thereof, is non-covalently associated with the article. In other embodiments, the inorganic or organic selenium compound, or formulation thereof, is covalently associated with the article.

In still further particular embodiments, the article is an adhesive tape or bandage. In yet other particular embodiments, the article is a cohesive tape or bandage.

In yet further embodiments, the surface of the article having the inorganic or organic selenium compound, or formulation thereof, further includes a pressure-sensitive adhesive. In particular embodiments, the pressure-sensitive adhesive is an acrylic water-based pressure-sensitive adhesive. In further embodiments, the pressure-sensitive adhesive is a hot-melt adhesive.

In particularly useful embodiments, at least about half of the elemental selenium from the inorganic or organic selenium compound, or formulation thereof, exists in an active state that is capable of generating superoxide radicals on the surface of the antimicrobial adhesive or cohesive article. In further particular embodiments, the article has at least one surface having at least about 2 ug of elemental selenium per square centimeter of surface area. In further useful embodiments, the article has at least one surface having at least about 6 ug of elemental selenium per square centimeter of surface area.

In a further aspect, the invention provides antimicrobial articles having, on at least one surface, an effective amount of an inorganic or organic selenium compound, or formulation thereof, capable of generating superoxide radicals in the presence of an infectious agent. The inorganic or organic selenium compound is non-covalently associated with the article and an effective amount of the inorganic or organic selenium compound, or formulation thereof, is retained on the surface of the article when the article is in cutaneous contact with a subject.

In another aspect, the invention provides an antimicrobial adhesive or cohesive article having, on at least one surface, an antimicrobial cohesive or adhesive formulation having an effective amount of an inorganic or organic selenium compound, or formulation thereof, capable of generating superoxide radicals in the presence of an infectious agent or reduced thiol compound. The inorganic or organic selenium compound is non-covalently associated with the article, and an effective amount of the inorganic or organic selenium compound, or formulation thereof, is retained on the surface of the article when the article is in cutaneous contact with a subject.

In a further aspect, the invention provides an antimicrobial adhesive or cohesive article product of the process of: providing an adhesive or cohesive formulation; adding to the adhesive or cohesive formulation an inorganic or organic selenium compound, or formulation thereof, capable of generating superoxide radicals in the presence of an infectious agent to produce an antimicrobial adhesive or cohesive formulation; and applying the antimicrobial adhesive or cohesive formulation to at least one surface of the article to produce the antimicrobial adhesive or cohesive article. The resulting antimicrobial adhesive or cohesive article carries, on at least one surface, an effective amount of the inorganic or organic selenium compound, or formulation thereof, that is capable of generating superoxide radicals in the presence of an infectious agent.

In yet another aspect, the invention provides an antimicrobial adhesive or cohesive article having, on at least one surface, an antimicrobial cohesive or adhesive formulation having an effective amount of an inorganic or organic selenium compound, or formulation thereof, capable of generating superoxide radicals in the presence of an infectious agent or a reduced thiol compound. The inorganic or organic selenium compound is covalently associated with the article and an effective amount of the inorganic or organic selenium compound, or formulation thereof, is retained on the surface of the article when the article is in cutaneous contact with a subject.

In particularly useful embodiments of the antimicrobial articles of the invention, the effective amount of the inorganic or organic selenium compound, or formulation thereof, that is retained on the surface of the article when the article is in cutaneous contact with the subject is sufficient to inhibit or inactivate an agent of infectious disease.

In certain embodiments of the above antimicrobial articles of the invention, the agent of infectious disease that is being treated or prevented is a bacteria. In particular embodiments the bacteria is Staphylococcus aureus, Pseudomonas aeruginosa, or Acinetobacter baumanii. In further embodiments of the invention, the agent of infectious disease is a fungus, a protozoa, or a virus.

In other embodiments, the inorganic or organic selenium compound, or formulation thereof, does not comprise a thiol group or a thiol-containing compound. In particular embodiments, the inorganic or organic selenium compound, or formulation thereof, does not comprise glutathione.

In further embodiments, the subject in which infectious disease is treated or prevented is a mammal or a marsupial. In particular embodiments, the mammal a mouse, a rat, a dog, a cat, a cow, a horse, a goat, a pig, a kangaroo or a yak. In other embodiments the subject is a human.

In certain particularly useful embodiments, the inorganic or organic selenium compound is selected selenite, selenate, selenocystine or selenomethionine. In other embodiments, the selenium compound is an organoselenium carboxylic acid compound, or alkyl ester thereof, such as selenocyanatoacetic acid, 3-selenocyanatopropionic acid, 2-selenocyanatopropionic acid, diselenocyanatodiacetic acid, diselenocyanatodipropionic acid, or mixtures thereof. In still other embodiments, the selenium compound is an organoselenium compound such as selenocyanatoethyl amine, 3-selenocyanatopropyl amine or selenocystamine dihydrochloride. In still other useful embodiments, the selenium compound is an organoselenium compound such as selenodicarboxylic acid, organoselenium diamines, organoselenium monoamines (and amides thereof), organoselenium amines (e.g., cystamine), selenoamino acids (e.g., L-selenocystine), organoselenium carboxylic acids (and amides thereof), selenourethanes, selenoureas, and selenofatty acids (and esters and amides thereof), selenochloroprene, selenobromoprene, selenostyrene, selenobutadiene, selenoacrylonitrile, or a mixture of one or more of these compounds. In still other embodiments, the selenium compound is a seleno adduct of a fatty acid, or an alkyl ester thereof. In further embodiments, the selenium compound is a polymer, or mixture of polymers, of an organoselenium compound or compound family such as selenoamino acids, 2-substituted organoselenium oxazolines, 2-substituted organoselenium oxazines, selenourethane, selenourea, selenochloroprene, selenobromoprene, selenostyrene, selenobutadiene, or selenoacrylonitrile.

In further particular embodiments, the inorganic or organic selenium compound, or formulation thereof, is non-covalently associated with the article. In other embodiments, the inorganic or organic selenium compound, or formulation thereof, is covalently associated with the article.

In still further particular embodiments, the article is an adhesive tape or bandage. In yet other particular embodiments, the article is a cohesive tape or bandage.

In yet further embodiments, the surface of the article having the inorganic or organic selenium compound, or formulation thereof, further includes a pressure-sensitive adhesive. In particular embodiments, the pressure-sensitive adhesive is an acrylic water-based pressure-sensitive adhesive. In further embodiments, the pressure-sensitive adhesive is a hot-melt adhesive.

In particularly useful embodiments, at least about half of the elemental selenium from the inorganic or organic selenium compound, or formulation thereof, exists in an active state that is capable of generating superoxide radicals on the surface of the antimicrobial adhesive or cohesive article. In further particular embodiments, the article has at least one surface having at least about 2 ug of elemental selenium per square centimeter of surface area. In further useful embodiments, the article has at least one surface having at least about 6 ug of elemental selenium per square centimeter of surface area.

In a final aspect, the invention provides for the substitution of tellurium (Te) for selenium (Se) in any and all of the above-described aspects and embodiments of the invention. In particular, while again not wishing to be bound by a single theoretical mechanism of action, it is believed that inorganic and organic tellurium compounds possesses the same microbial thiol/reducing agent -dependent and superoxide-based antimicrobial activity as does selenium. Accordingly, tellurium may be substituted for selenium in any of the aspects and embodiments of the invention described herein.

### 3. DESCRIPTION OF THE FIGURES

FIG. 1 is a graphical representation of the antimicrobial properties of an organotellurium and an organoselenium compound against MRSa (Methicillin-resistant Staphylococcus aureus).

### 4. DETAILED DESCRIPTION OF THE INVENTION

The patent and scientific literature referred to herein establishes knowledge that is available to those of skill in the art. The issued U.S. patents, allowed applications, published foreign applications, and references, which are cited herein are hereby incorporated by reference to the same extent as if each was specifically and individually indicated to be incorporated by reference.

4.1 General

In general, the invention provides organotellurium compounds, as well as inorganic and organic selenium compounds, formulations thereof, and associated organotellurium-carrying and selenium-carrying articles and methods for topical/cutaneous use in treating or preventing an agent of infectious disease such as a bacteria, a virus, a fungus, or a protozoa. The details of the proposed chemical mechanism of superoxide formation by selenite, and selenium's proposed involvement in toxicity and carcinostatic activity in vivo, has been reviewed by Spallholz ((1994) Free Radical Biology & Medicine 17: 45-64; see also Yan and Spallholz (1993) Biochemical Pharmacology 45(2): 429-436, and Mugesh and Singh (2000) Chem. Soc. Rev. 29: 347-357)), the contents of all of which are incorporated by reference herein in its entirety.

Applicants, having found potent antimicrobial activity in organotellurium compounds, have established that organotellurium compounds provide a similar catalytic activity for the formation of superoxide (O₂⁻) in the presence of reducing compounds such as the thiols present on the surface of an infectious microbe.

A review of the basic physical and chemical properties of tellurium and selenium is available (see (1991) Metals & Minerals, Annual Review), and a description of some of the basic biological properties of Selenium (Oldfield (1991) “Some Safety Considerations Involving Selenium” The Bulletin of Selenium-Tellurium Development Association, Inc., September ed., pp. 1-3; Yan et al. (1991) Biol. Trace Element Res. 30: 145-162; Shenberg et al. (1994) Biol. Trace Element Res. 40: 137-149; Spallholz (1994) Free Rad. Bio. & Med. 17: 45-64; and Soranio-Garcia (2004) Curr. Med. Chem. 11: 1657-1669) and Tellurium (Lamer (1996) “Tellurium in Health and Disease” The Bulletin of Selenium-Tellurium Development Association Inc., September ed., pp. 1-2) have also been described.

4.2 Organotellurium Compounds

The invention provides organic tellurium compounds, and formulations thereof, that are capable of generating superoxide radicals in the presence of an infectious agent, for use in the methods and incorporation into the articles and formulations of the invention.

Exemplary superoxide-forming organotellurium compounds include the following compounds. In general, R and/or R′ shown in each of the following structures can be any suitable Te-carrying carbon backbone that is capable of generating superoxide upon reacting with the sulfhydryl groups, including, but not limited to, —(C₁-C₅ alkyl)-, —O-(C₁-C₅ alkyl)-O—, —C₁-C₁₀ alkyl-, —O—(C₁-C₁₀ alkyl)-O—, —(C₂-C₁₀ alkenyl)-, —O—(C₂-C₁₀ alkenyl)-O—, -aryl, —C(O)OH, —C(O)O(C₁-C₅ alkyl), —C(O)O(C₁-C₅ alkyl)-O(O)C—, —OC(O)(C₁-C₅ alkyl), —OC(O)(C₁-C₅ alkyl)-(O)CO—, —NHC(O)(CH₂)ₙ—NHC(O)—, —NH₂, —NH—, —C(O)NH(CH₂)ₙ—NC(O)—, -halo, —OH, —O—, —CN, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), and -(7- to 10-membered bicyclic heterocycle).

The organoselenium compounds for use in the invention include:

Tellurodicarboxylic acids:

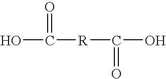

or alkyl esters thereof:

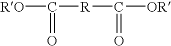

where Te can be contained within the R and/or R′ groups.

Tellurodiamines:
H₂N—R—NH₂

where R contains Te.

Ditelluroamines:
H₂NCH₂CH₂Te—TeCH₂CH₂NH₂

and others (e.g., cystamine)

Telluroamino Acids, e.g., L-Tellurocystine:

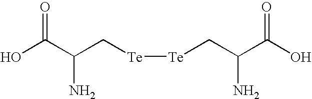

and others

Telluroamides made from organotelluriumamines:
NH₂—CH₂—R

tellurocarboxylic acids:

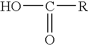

wherein Te is contained within the R groups.

Telluropolyamides (peptides) made from telluro amino acids
polymers made from Telluro dicarboxylic acids
polymers made from Telluro diamines or amino ditellurides
polymers made from ring opening polymerization of:
   a) 2-substituted oxazolines 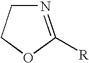
   b) 2-substituted oxazines 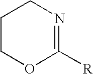
and polymer blends thereof
and Interpenetrating Polymer Networks (IPN(s)) thereof.

wherein the R groups above (a, b) contain a Te moiety

Tellurourethanes made from Te containing alcohols or isocyanates

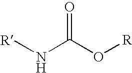

where R or R′ contains Te
and polymers thereof
and polymer blends thereof
and IPN(s) thereof
and Silane modified versions thereof

Ureas made from Te containing amines or isocyanates

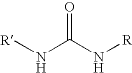

where R or R′ contain Te
and polymers thereof
and polymer blends thereof
and IPN(s) thereof
and Silane modified versions thereof

Te containing fatty acid esters of
Trymethylol propane
glycerol
penterythritol

Tellurium adducts of
chloroprene/bromoprene
styrene
butadiene
acrylonitrile
and polymers thereof
and polymer blends thereof
and IPN(s) thereof.
   4.3 Selenium Compounds

The invention further provides inorganic and organic selenium compounds, and formulations thereof, that are capable of generating superoxide radicals in the presence of an infectious agent, for use in the methods and incorporation into the articles and formulations of the invention.

Inorganic forms of selenium for use in the invention include selenium salts such as selenite (SeO₃⁻²), which are available and known in the art (e.g., sodium selenite (Na₂SeO₃) (available from Sigma-Aldrich, St. Louis, Mo.).

Exemplary superoxide-forming organoselenium compounds include the following compounds. In general, R and/or R′ shown in each of the following structures can be any suitable Se-carrying carbon backbone that is capable of generating superoxide upon reacting with the sulfhydryl groups, including, but not limited to, —(C₁-C₅ alkyl)-, —O—(C₁-C₅ alkyl)-O—, —C₁-C₁₀ alkyl-, —O—(C₁-C₁₀ alkyl)-O—, —(C₂-C₁₀ alkenyl)-, —O—(C₂-C₁₀ alkenyl)-O—, -aryl, —C(O)OH, —C(O)O(C₁-C₅ alkyl), —C(O)O(C₁-C₅ alkyl)-O(O)C—, —OC(O)(C₁-C₅ alkyl), —OC(O)(C₁-C₅ alkyl)-(O)CO—, —NHC(O)(CH₂)ₙ—NHC(O)—, —NH₂, —NH—, —C(O)NH(CH₂)ₙ—NC(O)—, -halo, —OH, —O—, —CN, —C₂-C₁₀ alkynyl, —C₃-C₈ monocyclic cycloalkyl, —C₈-C₁₄ bicyclic cycloalkyl, —C₅-C₈ monocyclic cycloalkenyl, —C₈-C₁₄ bicyclic cycloalkenyl, -(nitrogen-containing 3- to 7-membered monocyclic heterocycle), -(nitrogen-containing 7- to 10-membered bicyclic heterocycle), -(3- to 7-membered monocyclic heterocycle), and -(7- to 10-membered bicyclic heterocycle). Facile synthesis of these compounds is known in the art or can be achieved without undue experimentation through practice of the invention as described in the examples below (see, e.g., Jacob et al. (2004) Molecules pp. 622-626).

The organoselenium compounds for use in the invention include:

Selenodicarboxylic acids:

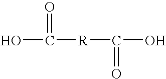

or alkyl esters thereof:

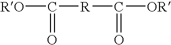

where Se can be contained within the R and/or R′ groups.

Selenodiamines:
H₂N—R—NH₂

where R contains Se.

Diselenoamines:
H₂NCH₂CH₂Se—SeCH₂CH₂NH₂

and others (e.g., cystamine)

Selenoamino Acids, e.g., L-Selenocystine:

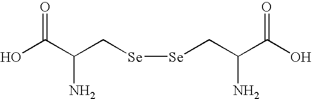

and others

Selenoamides made from organoseleniumamines:
NH₂—CH₂—R

selenocarboxylic acids:

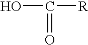

wherein Se is contained within the R groups.

Selenopolyamides (peptides) made from seleno amino acids
polymers made from Seleno dicarboxylic acids
polymers made from Seleno diamines or amino diselenides
polymers made from ring opening polymerization of:
   a) 2-substituted oxazolines 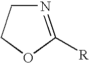
   b) 2-substituted oxazines 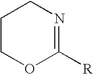
and polymer blends thereof
and Interpenetrating Polymer Networks (IPN(s)) thereof.

wherein the R groups above (a, b) contain a Se moiety

Selenourethanes made from Se containing alcohols or isocyanates

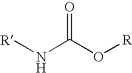

where R or R′ contains Se
and polymers thereof
and polymer blends thereof
and IPN(s) thereof
and Silane modified versions thereof

Ureas made from Se containing amines or isocyanates

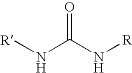

where R or R′ contain Se
and polymers thereof
and polymer blends thereof
and IPN(s) thereof
and Silane modified versions thereof

Se containing fatty acid esters of
Trymethylol propane
glycerol
penterythritol

Selenium adducts of
chloroprene/bromoprene
styrene
butadiene
acrylonitrile
and polymers thereof
and polymer blends thereof
and IPN(s) thereof.
   4.4 Organo-Te and Organo-Se Compounds and Adducts

In further defining the chemical nature of telluro-containing and seleno-containing R and/or R′ groups as detailed above, it is noted that:

The term “—C₁-C₅ alkyl” as used herein, refers to a straight chain or branched non-cyclic hydrocarbon having from 1 to 5 carbon atoms, wherein one of the hydrocarbon's hydrogen atoms has been replaced by a single bond. Representative straight chain —C₁-C₅ alkyls include -methyl, -ethyl, -n-propyl, -n-butyl and -n-pentyl. Representative branched —C₁-C₅ alkyls include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

The term “—C₁-C₆ alkyl” as used herein, refers to a straight chain or branched non-cyclic hydrocarbon having from 1 to 6 carbon atoms, wherein one of the hydrocarbon's hydrogen atoms has been replaced by a single bond. Representative straight chain —C₁-C₆ alkyls include -methyl, -ethyl, -n-propyl, -n-butyl, -n-pentyl and -n-hexyl. Representative branched —C₁-C₆ alkyls include -isopropyl, -sec-butyl, -isobutyl, -tert-butyl, -isopentyl, -neopentyl, -1-methylbutyl, -isohexyl, -neohexyl, -2-methylbutyl, -3-methylbutyl, -1,1-dimethylpropyl and -1,2-dimethylpropyl.

The term “—C₁-C₁₀ alkyl” as used herein, refers to a straight chain or branched non-cyclic hydrocarbon having from 1 to 10 carbon atoms, wherein one of the hydrocarbon's hydrogen atoms has been replaced by a single bond. Representative —C₁-C₁₀ alkyls include methyl, ethyl, propyl, butyl, pentyl, hexyl, heptyl, octyl, -nonyl, decyl, isopropyl, isobutyl, sec-butyl and tert-butyl, isopentyl, neopentyl, isohexyl, isoheptyl, isooctyl, isononyl and isodecyl.

The term “—(C₂-C₁₀)-alkenyl” as used herein, refers to a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “—(C₂-C₁₀) alkynyl” as used herein, refers to a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

The term “—(C₃-C₈) monocyclic cycloalkyl” as used herein, refers to a saturated cyclic hydrocarbon having from 3 to 8 carbon atoms. Representative (C₃—C₈)cycloalkyls include -cyclopropyl, -cyclobutyl, -cyclopentyl, -cyclohexyl, -cycloheptyl and -cyclooctyl.

The term “—(C₈-C₁₄) bicyclic cycloalkyl” as used herein, refers to a bi-cyclic hydrocarbon ring system having from 8 to 14 carbon atoms and at least one saturated cyclic alkyl ring. Representative —(C₈-C₁₄) bicycloalkyls include -indanyl, -1,2,3,4-tetrahydronaphthyl, -5,6,7,8-tetrahydronaphthyl, -perhydronaphthyl and the like.

The term “—(C₅-C₈) monocyclic cycloalkenyl” as used herein, refers to a cyclic non-aromatic hydrocarbon having at least one carbon-carbon double bond in the cyclic system and from 5 to 8 carbon atoms. Representative (C₄-C₈) monocyclic cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl and the like.

The term “—(C₈-C₁₄) bicyclic cycloalkenyl” as used herein, refers to a bi-cyclic hydrocarbon ring system having at least one carbon-carbon double bond in each ring and from 8 to 14 carbon atoms. Representative —(C₈-C₁₄) bicyclic cycloalkenyls include -indenyl, -pentalenyl, -naphthalenyl, -azulenyl, -heptalenyl, -1,2,7,8-tetrahydronaphthalenyl and the like.

A “nitrogen containing 3- to 7-membered monocyclic heterocycle” refers to a monocyclic 3- to 7-membered aromatic or non-aromatic monocyclic cycloalkyl group in which one of the cycloalkyl group's ring carbon atoms has been replaced with a nitrogen atom and 0-4 of the cycloalkyl group's remaining ring carbon atoms may be independently replaced with a N, o or S atom. The nitrogen containing 3- to 7-membered monocyclic heterocycles can be attached via a nitrogen, sulfur, or carbon atom. Representative examples of nitrogen-containing-3- to 7-membered monocyclic heterocycles include, but are not limited to, piperidinyl, piperazinyl, pyrrolyl, oxazinyl, thiazinyl, diazinyl, triazinyl, tetrazinyl, imidazolyl, tetrazolyl, pyrrolidinyl, isoxazolyl, pyridinyl, oxazolyl, thiazolyl, pyrazolyl, triazolyl, pyrimidinyl, morpholinium, and morpholinyl. In one embodiment, a nitrogen containing 3- to 7-membered monocyclic heterocycle is substituted with up to three groups, independently chosen from: —C₁-C₅ alkyl, -halo, -halo-substituted C₁-C₅ alkyl, hydroxy, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

Other selenium compounds for use in the invention are known in the art and described in, e.g., U.S. Pat. Nos. 4,512,977, 4,865,840, 5,922,346, 6,228,347, 6,303,651, and 6,867,23, the contents of which are incorporated herein by reference in their entirety.

Other organotellurium compounds for use in the invention are known in the art and described in, e.g., U.S. Pat. Nos. 4,076,530, 4,106,939, 4,144,062, 4,148,659, 4,152,155, 4,220,710, 4,271,090, 4,451,556, 4,613,468, 5,166,428, 5,759,760, and 7,026,228, the contents of which are incorporated herein by reference in their entirety.

4.5 Adhesive and Cohesive Formulations

The invention provides adhesive, including pressure-sensitive adhesives, as well as cohesive formulations into which the antimicrobial selenium compounds of the invention are incorporated.

Pressure-sensitive adhesives adhere to most surfaces with very slight pressure and they retain their tackiness. Pressure-sensitive adhesives include a large group of adhesives that utilize many different polymers (acrylics, rubbers, polyurethanes, silicones or siloxanes), together with plasticisers and tackifying resins to form a permanently tacky (sticky) adhesive. The name “pressure-sensitive” comes from the fact that moderate pressure alone is sufficient to spread the viscous adhesive layer on to the surface to be adhered to and achieve useful adhesive strength. They are available in both solvent and latex or water based forms. Pressure sensitive adhesives are often based on non-crosslinked rubber adhesives, acrylics or polyurethanes. They form viscoelastic bonds that are aggressively and permanently tacky, and adhere without the need of more than finger or hand pressure.

Generally, suitable pressure sensitive adhesives include, for example, those based on natural rubbers, synthetic rubbers, styrene block copolymers, polyvinyl ethers, poly (meth)acrylates (including both acrylates and methacrylates), polyurethanes, polyureas, polyolefins, and silicones. The pressure sensitive adhesive may comprise an inherently tacky material, or if desired, tackifiers may be added to a tacky or non-tacky base material to form the pressure sensitive adhesive. Useful tackifiers include, for example, rosin ester resins, aromatic hydrocarbon resins, aliphatic hydrocarbon resins, and terpene resins. Other materials can be added for special purposes, including, for example, plasticizers, hydrogenated butyl rubber, glass beads, conductive particles, filler, dyes, pigments, and combinations thereof.

Any pressure sensitive adhesive is useful for preparing the articles of the invention. Pressure-sensitive adhesives generally include elastomers that are inherently tacky or elastomers or thermoplastic elastomers that include tackifying resins and plasticizing additives. Fillers, antioxidants, stabilizers and crosslinking agents known in the art also may be used. A fluid, typically water, is added to reduce the viscosity to a level that is easily applied to the open fabric. The amounts and kinds of ingredients of the pressure-sensitive adhesive are selected to provide appropriate substrate adhesion and target peel strength. Strong substrate adhesion and a moderate peel strength are desired for use with living skin. Suitable pressure-sensitive adhesives include polyacrylate adhesives, polyalphaolefin adhesives, such as linear, radial, branched and tapered block copolymers including styrene-butadiene, styrene-ethylene/butylenes and styrene-isoprene block copolymers, polyvinyl acrylates, natural and synthetic rubber resin adhesives, silicones, polydiorganosiloxane polyurea copolymers, and mixture and blends thereof. Many suitable pressure-sensitive adhesives are known in the art and may be utilized with the subject invention. Particularly useful pressure-sensitive adhesives for use in the invention include acrylic resins (e.g., Gelva™ Multipolymer Solution 2495; Cytec Surface Specialties; Indian Orchard, Mass.).

The adhesive can be located on upper and/or lower surfaces of the article (e.g., an open fabric or a film). Where the article is or includes a fabric, the pressure-sensitive adhesive may cover optionally the upper and lower surfaces without spanning adjacent yarns, so that porosity or openness is retained. Where the article is or includes a film, the pressure-sensitive adhesive may cover either of both surfaces of the film. The adhesive may also be suffused or permeated throughout the entire thickness of the open fabric of an article. The pressure-sensitive adhesive may be selected to be removable from the skin without separation of the substrate backing from the open fabric.

Exemplary cohesive formulations for use in the invention are known in the art and described, e.g., in U.S. Pat. No. 6,156,424 (“Cohesive Products”). Exemplary adhesive formulation are also known in the art and described in, for example, U.S. Pat. No. 4,112,213, U.S. Pat. No. 4,917,928, U.S. Pat. No. 4,917,929, U.S. Pat. No. 5,141,790, U.S. Pat. No. 5,045,386, U.S. Pat. No. 5,229,207, U.S. Pat. No. 5,296,277, U.S. Pat. No. 5,670,557, U.S. Pat. No. 6,232,366, and U.S. Publication No. 2005/0249791, the disclosures of which as incorporated herein by reference in their entireties.

4.6 Antimicrobial Articles

The invention further provides articles for topical/cutaneous contact with a subject (e.g., tapes and bandages) onto which the antimicrobial selenium compounds of the invention are incorporated. Exemplary articles include tapes and bandages and may be constructed of any number of materials woven and non-woven fabrics, knit fabrics and films, including porous films (exemplary porous films are described in U.S. Ser. No. 11/204,736). The antimicrobial articles of the invention may be used in any suitable application, e.g., in sports or medicine. Exemplary articles for topical/cutaneous contact for use in the invention are known in the art and described, e.g., in U.S. Pat. No. 5,762,623 (“Elastic Bandage”), U.S. Patent Publication No. 20040214494 (“Stretch Fabric”), 20050158539 (“Pressure-Sensitive Adhesive Tapes”) and 2005/0249791 (“Antimicrobial Articles”) the contents of which are incorporated herein by reference in their entirety.

### 5. EXAMPLES

The following examples serve to illustrate certain useful embodiments and aspects of the present invention and are not to be construed as limiting the scope thereof. Alternative materials and methods can be utilized to obtain similar results.

### Example 1

### Preparation of Selenocystamine dihydrochloride (2,2′-diselenodiethanamine dihydrochloride)

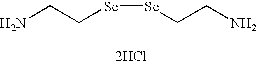

To a 3-neck flask fitted with an N₂ gas inlet, an addition funnel, and a gas outlet leading to a trap containing a 5% Pb(OAc)₂ solution, was added selenium metal (4.5 gm, 56.9 mmol), followed by 25 mL H₂O. In a separate flask, NaBH₄ (4.5 gm, 118.5 mmol) was taken up in 25 mL H₂O and the solution was transferred to the addition funnel. The selenium metal slurry at room temperature was stirred at a moderate rate and the NaBH₄ solution was slowly added drop wise at such a rate to keep the gas evolution and effervescence at a moderate pace. The solution changed from grey to brown-red and finally to a clear light yellow after complete addition (approximately 50 min.). After 10 minutes a second amount of selenium metal (4.5 gm, 56.9 mmol) was added in portions. The solution turned a dark red-brown color and was stirred for another 20 minutes at room temperature. A solution of 2-chloro-ethylamine hydrochloride (13.9 gm, 119.7 mmol) in 30 mL aqueous 20% sodium hydroxide was added to the reaction drop wise and stirring was continued at room temperature overnight (ca. 17 hours). The reaction was poured into a separatory funnel and extracted with CHCl₃. The combined CHCl₃ layers were dried over anhydrous MgSO₄, filtered and concentrated to yield 9.8 gm of a light yellow oil, consisting of a mixture of 2,2′-diselenodiethanamine and 2,2′-selenodiethanamine. This material was dissolved in 200 mL of methanol/ethyl acetate (1:1) and treated with anhydrous HCl in diethyl ether (1N solution). The solution was concentrated to yield a 10.1 gm of a light yellow solid. The solid was dissolved in methanol and ethyl acetate was slowly added until a precipitate started forming. Filtration, collection and drying of the solids produced 2.3 gm of 2,2′-diselenodiethanamine dihydrochloride as a light yellow powder.

### Example 2

### Preparation of 2,2′-tellurodiethanamine dihydrochloride

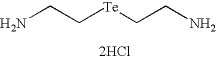

To a 3-neck flask fitted with an N₂ gas inlet, an addition funnel and a gas outlet leading to a trap containing a 5% Pb(OAc)₂ solution, was added tellurium metal (10 gm, 78.4 mmol), followed by 250 mL H₂O. The resulting slurry was de-oxygenated with N₂ gas for 20 minutes. In a separate flask, NaBH₄ (7.7 gm, 203.8 mmol) was taken up in 90 mL H₂O, transferred to the addition funnel and the solution was de-oxygenated with N₂ gas for 20 minutes. The tellurium metal slurry was heated to 65-70° C. and stirred at a moderate rate. The NaBH₄ solution was slowly added drop wise at such a rate as to keep the gas evolution and effervescence at a moderate pace. The solution changed from graphite to metallic silver to dark purple to light pink and finally formed a clear, colorless solution after complete addition. After 20 minutes, the clear solution was cooled in an ice bath. A solution of 2-bromo-ethylamine hydrobromide (35.3 gm, 172.5 mmol) in 100 mL aqueous 20% sodium hydroxide was de-oxygenated with N₂ gas and added to the reaction drop wise. The ice bath was removed and the reaction stirred at room temperature for 4.5 hours. The reaction was poured into a separatory funnel and extracted with CHCl₃ (4×200 mL). The combined CHCl₃ layers were dried over anhydrous MgSO₄, filtered and concentrated to yield 10.2 gm of a red/yellow oil, consisting of crude 2,2′-tellurodiethanamine. This material was dissolved in 400 mL of methanol/ethyl acetate (1:1) and treated with anhydrous HCl in diethyl ether (2N solution). The solution was concentrated to yield 13.4 gm of 2,2′-tellurodiethanamine dihydrochloride as a light yellow powder.

### Example 3

### Preparation of 2,2′-ditellurodiethanamine dihydrochloride

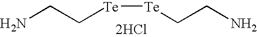

To a 3-neck flask fitted with an N₂ gas inlet, an addition funnel and a gas outlet leading to a trap containing a 5% Pb(OAc)₂ solution, was added tellurium metal (4.0 gm, 31.4 mmol), followed by 100 mL H₂O. The resulting slurry was de-oxygenated with N₂ gas for 20 minutes. In a separate flask, NaBH₄ (3.1 gm, 81.5 mmol) was taken up in 36 mL H₂O, transferred to the addition funnel and the solution was de-oxygenated with N₂ gas for 20 minutes. The tellurium metal slurry was heated to 65-70° C. and stirred at a moderate rate. The NaBH₄ solution was slowly added drop wise at such a rate as to keep the gas evolution and effervescence at a moderate pace. The solution changed from graphite to metallic silver to dark purple to light pink and finally formed a clear, colorless solution after complete addition. Upon formation of the clear solution, a second amount of tellurium metal (4.0 gm, 31.4 mmol) was added in portions, resulting in the formation of a deep purple solution. The slurry was heated for 4 hours at 65-70° C., and the solution was cooled in an ice bath. A solution of 2-bromo-ethylamine hydrobromide (14.1 gm, 68.9 mmol) in 41 mL aqueous 20% sodium hydroxide was de-oxygenated with N₂ gas and added to the reaction drop wise. The ice bath was removed and the reaction stirred at room temperature for 2 hours. The reaction was poured into a separatory funnel and extracted with CHCl₃ (3×100 mL). The combined CHCl₃ layers were dried over anhydrous MgSO₄, filtered and concentrated to yield 5.7 gm of a dark red oil, consisting of a mixture of 2,2′-ditellurodiethanamine and 2,2′-tellurodiethanamine. This material was dissolved in 120 mL of methanol/ethyl acetate (1:1) and treated with anhydrous HCl in diethyl ether (2N solution). The solution was concentrated to yield 6.9 gm of a dark yellow solid. The solid was dissolved in methanol and ethyl acetate was slowly added until a precipitate started forming. Filtration, collection and drying of the solids produced 1.2 gm of 2,2′-ditellurodiethanamine dihydrochloride as a light orange powder.

### Example 4

### Preparation of 2-Selenocyanatoethyl Methacrylate

H₂C═C(CH₃)—C(═O)—O CH₂ CH₂—SeCN

A solution of 2-bromoethyl methacrylate (9.65 g; 50 mmol) in 200 mL of THF was added drop-wise to a stirring solution of 8.0 g (55 mmol) of potassium selenocyanate in 200 mL of THF. Immediately upon reaction, a white precipitate of potassium bromide formed. When the addition was completed, the reaction mixture was heated to reflux, for about 20 min., and then allowed to return to room temperature.

The potassium bromide precipitate is removed by gravity filtration and then washed twice with diethyl ether. The diethyl ether fractions are combined with the THF filtrate and then dried over magnesium sulfate. The magnesium sulfate is removed by gravity filtration and the filtrate is then concentrated under reduced pressure by rotary evaporation. The resultant residue represents greater than 75% yield of 2-selenocyanatoethyl methacrylate.

### Example 5

### Method for the Fluorometric Micro-Determination of Selenium

This example describes a fluorescence-based assay used to measure the total amount of selenium in a sample. The same assay may be used to measure the total amount of tellurium in a sample for organotellurium-based applications of the invention.

Standard Selenium Solution

A stock Se solution (1000 ng Se/ml) was prepared from dried sodium selenite by dissolving the Se salt in analytical-grade water. From this stock solution 100 ng Se/ml and other standard Se solutions were prepared by dilution.

Digestion Mixture

The digestion mixture used was that as described by Cummins et al. (1965) D. D. Anal. Chem., 37:430 (1965). Five grams of sodium molybdate were dissolved in 75 ml of water. 75 ml of concentrated sulfuric acid were slowly added to the molybdate solution. After cooling, 100 ml of 70-72% perchloric acid was added.

Diaminonaphthalene (DAN) Solution

Diaminonaphthalene solution was prepared according to the procedure described by Hoffman et al. (1968) J. Assoc. Off. Agric. Chem., 51:1039. The following solution was prepared just prior to use. Into a stirring solution of aqueous sulfuric acid (30 ml), 50 mg of 3,3′-diaminonaphthalene was dissolved. This solution was added to 140 ml of concentrated sulfuric acid and dilute to 1 liter. The resulting solution was transferred to a separatory funnel to which 50 ml of cyclohexane (spectrophotometric grade) were added and the resulting mixture was shaken for 10-15 min. After allowing time for separation of the phases, the lower sulfuric acid phase was drawn off for immediate use. (Note: This reagent is sufficient for 12 analyses using the single-test-tube method.) The DAN solution required a minimum of three washes with cyclohexane to remove interfering fluorometric material (the cyclohexane wash should be checked fluorometrically prior to using the DAN solution).

Disodium EDTA Solution

Dissolve sufficient disodium ethylenediaminetetraacetic acid hydrate in water and dilute to 500 ml (0.2 M). This solution was further diluted to prepare 0.008 M EDTA used in the method.

Additional Reagents and Equipment

All reagents were of analytical grade or better. Cyclohexane and sulfuric acid were spectrophotometric grade. Other equipment used included 19 mm by 150 mm Pyrex or Kimax test tubes with ground-glass tops or screw caps, 3-5 mm glass beads, Fisher burner, test-tube holder and rack, pH meter, large beaker and hot plate, thermometer, separatory funnel, pipettes and aspirator, analytical balance, centrifuge for 19 mm by 150 mm test tubes, and fluorometer with 4-ml fluorometric cell.

Procedure

Into 19 mm by 150 mm acid-cleaned Pyrex or Kimax test tubes with either ground-glass tops or screw caps, 0.10-0.50 g of sample (e.g., tape or bandage) was placed. Sufficient water was added to equal 1 g (1 ml) of sample. The Se standards were prepared likewise, as was a water blank in a total volume of 1 ml. 2-3 acid-washed glass beads (3-5 mm) were added to 2 ml of the digestion mixture. Over a Fisher burner samples and standards were individually brought to a boil and digestion was continued until white fumes were driven off and the sample volume has been reduced to approximately 1 ml. The digestion mixture should be free of carbon at this point and will be briefly yellow in color. (Caution: Safety glasses and protective clothing should be worn as organic material will react vigorously with the hot digestion mixture.)

Following digestion the samples were diluted to 7 ml with 0.008 M EDTA. At this time all samples were, and should be, colorless (otherwise the digestion is incomplete). To each test tube 2.1-2.2 ml of concentrated NH₄OH was added. Using a pH meter, pH was adjusted to 2 in the test tube using either concentrated NH₄OH or H₂S0₄. Only a very few drops of either acid or base were (and should be) required. To each sample 4 ml of freshly prepared DAN reagent was added (prior to use, the cyclohexane wash was checked to confirm removal of interfering fluorometric material from the DAN solution). The resulting solution was mixed well, and incubated in a water bath at 50° C. for 20 mm. After incubation, the tubes were cooled for 5 min and 4 ml of cyclohexane was added. The tubes were then capped and shaken for 5 min. The glass stoppers were removed and the tubes were centrifuged for 5 min. The cyclohexane was then directly transferred to the fluorometric cuvette. Fluorescence from the 4,5-benzopiazselenol complex in each sample was then quantitated by excitation at 363 nm with the emission measured at 525 nm having been zeroed against a chloroform blank. Data were plotted as relative fluorescence intensity against a 1 ng Se/4 ml of cyclohexane standard.

### Example 6

### Chemiluminescent (CL) Assay

This example describes the chemiluminescence-based assay used to measure the amount of reactive selenium available in a sample for thiol-dependent superoxide formation. The same assay may be used to measure the total amount of reactive tellurium available in a sample for thiol-dependent superoxide formation for organotellurium-based applications of the invention.

The control chemiluminescent (CL) assay cocktail without substrates or GSH was made using a 0.05 M sodium phosphate buffer (pH 7.0) and 20 μL lucigenin/mL from a stock solution of 1.0 mg/mL lucigenin in distilled water. The assay cocktail with thiol contained 1.0 mg GSH/mL. To 600 μL test aliquots of the control or thiol containing assay cocktail was added L-selenomethionine or L-Se-methyl-selenocysteine at 2.0 mg/mL and other substrate concentrations were made by dilution with buffer containing lucigenin. In like manner, D, L-selenoethionine was added at 4.0 mg/mL to the CL cocktail. To the control or substrate-containing CL cocktails in the luminometer was added methioninase containing 0.5 U of enzyme activity or graded units of methioninase activity. Methioninase was prepared by adding 1.0 or 2.0 mL of distilled water to 10 U vials of commercial freeze-dried enzyme. The methioninase was reported by Waco technical services to contain no reducing thiol preservative. The enzyme was added in 0.1 mL increments from a 1.0 cm³ syringe or up to 30 μL from an Eppendorf pipette directly to the chemiluminescent tube in a Los Alamos Diagnostics Model 535 luminometer containing 600 μL of the pH 7.0 cocktail. The CL tube contents was held at 36.8° C. by an attached LKB 2209 multitemp recirculating water bath. Chemiluminescent (CL) data was recorded in 30-s integrated units over a period of up to 20 min. There was a 3-s instrumental delay between integrations. Additional details of this assay, including the quenching of chemiluminescence generated by methylselenol from reduced methylseleninic acid and dimethyldiselenide by superoxide dismutase has been previously reported. This CL assay is quantitative (correlation coefficient, r ¼ 0:99; P<0:001) in generating CL for small amounts of redox cycling methylselenol. A standard curve for methylselenol (CH₃SeH) produced CL (relative CL units vs. selenium concentration) from the reduction of dimethyldiselenide by GSH when added directly to the CL cocktail, can be used to facilitate quantitation of the experimental samples.

### Example 7

### X-RAY Fluorescence (XRF) Detection of Se and Te on Surfaces

The following method is a useful non-destructive, ultra sensitive detection and differentiation tool for determining the amount of bioavailable Tellurium or Selenium on the surface of an antimicrobial article. Analyzed samples may be used for further testing without waste or interference. XRF is an essential tool for the understanding of bioavailability and is not only able to determine the concentration of Se or Te at the surface but also the homogeneity of distribution across the surface. This tool is critical in the research and development of formulated products and their drying profiles to optimize conditions and chemistries to enhance migration of the bioactive compounds to the surface where they are available to interact with and kill even resistant and pervasive nosocomial pathogens present.

100 mg of Selenocystamine dihydrochloride was added to 19.9g of water-based PSA with agitation. 8 g-12 g of this coating was drawn down on a tape substrate, flash dried for 2 min, heated at 115° C. for 1.5 min. and the samples were cut into 1″×4″ strips, weighed and were analyzed using a Niton rapid, hand-held, point and shoot XRF analyzer to determine the amount of selenocystamine which has “blushed” to the surface and how it is distributed across the sample.

### Example 8

### Bioavailability of Se vs Coating Open Time

The following method demonstrates that drying conditions for organoselenium and organotellurium formulations (e.g., PSA formulations) of the invention can be adjusted to optimize the bioavailability as a function of coating open time.

The samples were made from the same batch of bioactive PSA and coated independently affording only slight variations in total Selenium present. Only the drying conditions of temperature and dwell time were varied from this experiment. For the first experiment, experiment A, one gram of (Phenylselenomethyl)trimethylsilane was added to 47 g of an acrylic PSA (pressure-sensitive adhesive) in 2 g ethyl acetate formulated PSA. The formulated PSA was drawn down onto a suitable substrate such as Mylar and then subjected to drying conditions designed to determine the affect of coating open time on the bioavailability of the selenium in the sample. In particular, a sample of Mylar coated with 50-55 g/m² of bioactive PSA was dried at 115° C. for 1.5 min. The total Se concentration in this sample was 39.2 μg/cm² and the bioavailability of the Se, as determined by X-Ray Fluorescence at the surface was 26.76 μg/cm² or 68.2% of the total Selenium in the sample. For the second experiment, experiment B, a sample of Mylar coated from the same batch of bioactive PSA @50-55 g/m² was dried at 93° C. for 2.0 min. The total concentration of Selenium in the sample as determined by weight was 38 μg/cm² and the bioavailability of the Se, as determined by X-Ray Fluorescence at the surface was 33.10 μg/cm² or 87% of the total Selenium in the sample.

The significant increase in Selenium bioavailability in experiment B is due to the increased coating open time afforded by the increased dwell time at lower temperature.

### Example 9

### Solution-Based Assay for Antimicrobial Activity

In this example, a test method is used for the determination of the kinetics of microbial killing or inactivation. The protocol follows that of ASTM (American Society for Testing and Materials) protocol E 2315-03 and is briefly summarized below.

The test material (e.g., organotellurium, organoselenium or inorganic selenium compound) or a dilution of the test material is brought into contact with a known population of microorganisms for a specified period of time at a specified temperature. The test material is separated from the population of microorganisms at appropriate sampling intervals (for example: 30 s, 60 s, or any range covering several minutes or hours) using appropriate techniques (e.g., sedimentation and washing of the microorganisms). The test material is neutralized at the sampling time and the surviving microorganisms are then counted. The percent, or log₁₀ reduction, or both, from either an initial microbial population or from a test blank is then calculated.

This procedure may be used to assess the in vitro reduction of a microbial population of test organisms after exposure to a test material.

Test organisms are representative of standardized strains of Staphylococcus aureus, Pseudomonas aeruginosa, Klebsiella pneumonia and Acinetobacter baumannii and capable of providing reproducible results under specific test conditions. Appropriate strains of these and other test microbes are available publicly, e.g. from the ATCC (American Type Culture Collection, O. Box 1549, Manassas, Va.). The cultures are transferred from stock twice (once every 18-24 h as appropriate for the test organism) into the appropriate growth media. The second transfer may be made into a volume of growth medium to produce sufficient microbial suspension to inoculate. Volumes used should permit testing of multiple samples or time points.

Alternatively, the transfers are made onto agar plates (or slants) and the inoculum suspension is prepared by washing the organism from the slant or plate with an appropriate broth or diluent.

The volume of inoculum suspension should be kept less than or equal to 5% of the total volume of the test volume. The microbial population or numbers control should contain a minimum of 10⁶ cfu/mL test material.

The test sample is mixed and the appropriate inoculum suspension is added to the sample or to the control blank and mixing is maintained to disperse the inoculum suspension. The inoculum suspension should be uniformly mixed. Maintaining uniform mixing throughout the test is critical for test repeatability. Where applicable, mix carefully to minimize foam formation. The formation of foam may cause anomalous results.

At predetermined time intervals, an aliquot (usually 1 mL) is removed from the sample/inoculum container and the appropriate dilution is made in sterile Butterfield's buffered phosphate diluent or equivalent containing appropriate neutralizers, if needed.

Then, recover viable organisms from the appropriate dilutions by culturing in duplicate by use of spread- or pour-plating, microbial filtration, spiral plating, or other valid microbial recovery methods.

Plates are incubated at the specified temperature ±2° C. for 24 to 48 h or as appropriate for each organism selected. Incubation time should allow for the growth of surviving organisms, without overgrowth of colonies, making enumeration difficult.

To determine surviving organisms, colonies are counted and the raw data is recorded as cfu/plate. The duplicate plates (2 plates from each replicate dilution) are averaged and then multiplied by the dilution factor to afford cfu/mL test suspension. This averaged count is then converted into log₁₀.

### Example 10

### Plate-Based Disk Diffusion Assay for Antimicrobial Activity

In this example, a test method is used for the determination of the properties of microbial killing or inactivation by an organotellurium or organoselenium or inorganic selenium compound, or formulation thereof. The protocol follows the disk diffusion susceptibility assay protocol that has been used to characterize antibiotic action of ampicillin and other antibiotic agents. This assay has been described in great detail elsewhere (see, e.g., Lennette, E. H., A. Balows, W. J. Hausler, Jr., and H. J. Shadomy (ed). (1985) Manual of clinical microbiology, 4th ed. American Society for Microbiology, Washington, D.C.; Washington, J. A. (ed.). (1981) Laboratory procedures in clinical microbiology, Springer-Verlag, New York, N.Y.; National Committee for Clinical Laboratory Standards, (1984) Performance standards for antimicrobial disk susceptibility tests, NCCLS Approved Standard M2-T3, Villanova, Pa.; National Committee for Clinical Laboratory Standards (1987) Performance standards for antimicrobial susceptibility testing) and is briefly summarized below. In general, the example shows the spatially-limited antimicrobial action of the test tellurium or selenium compounds, which prevent growth up to and underneath the test disk to which they are applied.

The purpose of this assay is to determine the antimicrobial susceptibility pattern of a bacterial strain. Suspension of the bacterial strain to be tested is adjusted to a standard density, and the suspension is swabbed evenly on a Mueller-Hinton agar plate. Antimicrobial disks are applied to the inoculated surface of the Mueller-Hinton plate. When bacterial multiplication proceeds more rapidly than the drug can diffuse, the bacterial cells that are not inhibited by the antimicrobial agent will continue to multiply until a lawn of growth is visible and no zone of inhibition appears around the disk. When the antimicrobial agent is present in inhibitory concentrations, no growth will appear in the zone around the disk. The more susceptible the bacterial strain tested, the larger the zone of inhibition. The diameter of the zone of inhibition is indirectly proportional to the minimal inhibitory concentration (MIC). The approximate MICs that correspond to the “susceptible,” “moderately susceptible,” “intermediate,” and “resistant” categories are listed in Zone Diameter Standards for Antibiotic Susceptibility Procedure:
A. Select 3 to 5 isolated colonies. Touch the top of each colony with a sterile inoculation loop or a sterile swab and inoculate a tryptic soy broth tube (5 ml) or Mueller-Hinton broth (5 ml).
B. Allow the broth culture to incubate at 35-37° C. until it achieves or exceeds the turbidity of the McFarland No. 0.5 standard. If the turbidity exceeds the standard, adjust the turbidity of the broth culture with tryptic soy broth to obtain a turbidity that visually matches the tubidity of the No. 0.5 McFarland standard. To perform this step, use adequate light, and read the tube against a white background with contrasting black lines.
C. After adjusting the turbidity of the inoculum suspension, dip a sterile swab into the suspension and rotate the swab several times with a firm pressure on the inside wall of the tube above the fluid level to remove excess inoculum from the swab.
D. Inoculate the surface of a Mueller-Hinton agar plate by streaking the swab over the entire agar surface in three different directions to ensure an even distribution of the inoculum. If the plate is satisfactorily streaked, the zone of inhibition will be uniformly circular, and there will be a uniformly confluent lawn of growth.
E. As soon as the plate surface dries, place the appropriate drug-impregnated disks on the surface of the inoculated agar plate. Use the disk dispenser provided for this purpose. With forceps, gently press down each disk to ensure complete contact with the agar surface. Since some of the drug diffuses almost instantaneously, a disk should not be removed once it has contact with the agar surface.
F. Invert the plates and place them in a 35-37° C. non-CO₂ incubator within thirty minutes after the disks are applied.
G. After 18-24 hours of incubation, examine plates and measure the diameters of the zones of inhibition to the nearest whole millimeter using a ruler held on the back of the petri plate. (Keep cover on the petri plate). An alternate method for determining if the broth culture has achieved the turbidity of the McFarland No. 0.5 standard is to use a spectophotometer. Place the tube in the spectophotometer and read the absorbance at 600 nm; the absorbance of the McFarland No. 0.5 standard is approximately 0.132. The preparation of McFarland standards is known in the art.

Interpretation

Interpret the sizes of the zones of inhibition by referring to Zone Diameter Standards for Antibiotic Susceptibility and report the organism to be either susceptible, moderately susceptible, intermediate, or resistant. A categorization of “susceptible” implies that an infection due to the organism tested may be expected to respond to the dosage of antimicrobial agent recommended for that type of infection and infecting species. Bacterial strains in the “resistant” category are not completely inhibited by concentrations within the therapeutic range. The moderately susceptible category includes strains that may be inhibited by concentrations of certain antimicrobial agents, e.g., beta-lactams, using high dosages or in body sites where drugs are physiologically concentrated. The intermediate category provides a “buffer zone” that prevents small uncontrolled technical factors from causing discrepancies in interpretations; results in this category may be considered equivocal, and if alternative drugs are not available, dilution tests may be indicated. The categories listed in the table were developed by comparing zone sizes with MIC values in broth and agar dilution tests. These categories were related to blood levels usually obtained with frequently used dose schedules or in the case of nitrofurantoin or nalidixic acid, with urine levels.

### Example 11

### Biofilm Assay for Antimicrobial Activity

The inhibition of biofilm formation from Staphylococcus aureus or Pseudomonas aeruginosa with the organoselenium coating is investigated by incubating the selenium-coated article and an uncoated control article in a nutrient broth containing either aforementioned strain of bacteria for four days at 37° C. The articles are rinsed with saline, fixed with glutaraldehyde and subsequently inspected using a scanning electron microscope to observe the presence of bacterial colonization.

Electron micrographs show little to no bacterial colonization on the selenium coated article but extensive colonization and biofilm formation on the uncoated control article using each of the aforementioned bacterial strains.

The same method is used with an organotellurium containing coating to demonstrate inhibition of bacterial growth and biofilm formation by organic tellurium compounds of the invention.

### Example 12

### Antimicrobial Activity of Organotellurium and Organoselenium Compounds

Tellurium is an element that occurs in the same column of the periodic table that selenium is in (i.e., Group 16), and therefore has a similar valence electron arrangement. In order to determine whether organotellurium compounds possess similar antimicrobial properties to those of organoselenium compounds, representative organotellurium and organoselenium compounds were prepared and their ability to kill (i.e., reduce the number of colony forming units (CFU)) of a population of MRSa (Methicillin-resistant Staphylococcus Aureus) was investigated.

Methicillin-resistant Staphylococcus Aureus (MRSA) is a type of bacteria that is resistant to certain antibiotics. These antibiotics include methicillin and other more common antibiotics such as oxacillin, penicillin and amoxicillin. Staph infections, including MRSA, occur most frequently among persons in hospitals and healthcare facilities (such as nursing homes and dialysis centers) who have weakened immune systems. MRSA infections that are acquired by persons who have not been recently (within the past year) hospitalized or had a medical procedure (such as dialysis, surgery, catheters) are known as CA-MRSA infections (Community-associated MRSA). Staph or MRSA infections in the community are usually manifested as skin infections, such as pimples and boils, and occur in otherwise healthy people.

Three organoseleno-/organotelluro-compounds were prepared using organic synthetic schemes, except where indicated otherwise, as reported further herein. The first compound, Agent 1 was selenocystamine dihydrochloride (C₄H₁₂N₂Se₂-2HCl, FW=318.9, amount=200 mg, tested at 82 mM) having the structure: HCl.H₂N—CH₂ CH₂—Se—Se—CH₂ CH₂—NH₂.HCl and the second compound, Agent 2, was the same compound (i.e., selenocystamine dihydrochloride having the same structure: HCl.H₂N—CH₂ CH₂—Se—Se—CH₂ CH₂—NH₂.HCl, tested at 41 mM), but obtained commercially from Sigma Chemical Company (St. Louis, Mo.). The third compound, Agent 3, was the organotellurium compound 2,2′-tellurodiethanamine 2 HCl (100 mg, MW=215.6, FW=288.6, tested at 47 mM) having the structure: HCl.H₂N—CH₂ CH₂—Te—CH₂ CH₂—NH₂.HCl.

A fourth compound, Agent 4, an aliphatic seleno methacrylic acid ester such as 2-selenocyanatoethyl methacrylate (500 mg dissolved in DMSO), is prepared and tested at 1, 10, and 100 mM concentrations.

Testing Methods

Analyzing the efficacy of antimicrobial agents may be performed by various suspension and susceptibility methods. This study is designed to examine the rate-of-kill of a test substance against sponsor selected pure cultures of microorganisms. This is accomplished by exposing the target microorganism(s) to the test substance and inspecting the solution for potential survivors at various time periods. The experimental design in this protocol meets these requirements.

A suspension of bacterial cells is exposed to the test substance for specified contact times. After exposure, an aliquot of the suspension is transferred to a neutralizer and assayed for survivors. Appropriate purity, sterility, microorganism population and neutralization controls are performed.

| | | | Incubation |
|---|---|---|---|
| Test Organism | ATCC # | Culture Medium | Parameters |
| Methicillin-Resistant | 33592 | Blood Agar | 35-37° C., |
| Staphylococcus aureus | | (Tryptic Soy Agar | aerobic |
| (MRSa) | | with 5% Sterile | |
| | | Sheep Blood) | |

Further MRSa test organism(s) to be used in this study can be obtained from the American Type Culture Collection (ATCC), Manassas, Va. Exemplary MRSa available from the ATCC include the following:

| ATCC ® Number | Description | Designation |
|---|---|---|
| 33591 | Staphylococcus aureus subsp. | 328 |
| | aureus | |
| 700787 | Staphylococcus aureus subsp. | 2947 |
| | aureus deposited as | |
| | Staphylococcus aureus | |
| | Rosenbach | |
| 700788 | Staphylococcus aureus subsp. | 406 |
| | aureus deposited as | |
| | Staphylococcus aureus | |
| | Rosenbach | |
| 700789 | Staphylococcus aureus subsp. | 12478 |
| | aureus deposited as | |
| | Staphylococcus aureus | |
| | Rosenbach | |
| BAA-42 | Staphylococcus aureus subsp. | HDE288 |
| | aureus deposited as | |
| | Staphylococcus aureus | |
| | Rosenbach | |
| BAA-43 | Staphylococcus aureus subsp. | HSJ216 |
| | aureus deposited as | |
| | Staphylococcus aureus | |
| | Rosenbach | |
| BAA-44 | Staphylococcus aureus subsp. | HPV107 |
| | aureus deposited as | |
| | Staphylococcus aureus | |
| | Rosenbach | |
| BAA-811 | Staphylococcus aureus | 308118L |
| 700698 | Staphylococcus aureus subsp. | Mu3 |
| | aureus deposited as | |
| | Staphylococcus aureus | |
| | Rosenbach | |
| BAA-41 | Staphylococcus aureus subsp. | NYBK2464 |
| | aureus deposited as | |
| | Staphylococcus aureus | |
| | Rosenbach | |
| 700699 | Staphylococcus aureus subsp. | Mu50 |
| | aureus deposited as | |
| | Staphylococcus aureus | |
| | Rosenbach | |
| BAA-40 | Staphylococcus aureus subsp. | CPS22 |
| | aureus deposited as | |
| | Staphylococcus aureus | |
| | Rosenbach | |
| BAA-38 | Staphylococcus aureus subsp. | E2125 |
| | aureus deposited as | |
| | Staphylococcus aureus | |
| | Rosenbach | |
| 43300 | Staphylococcus aureus subsp. | F-182 |
| | aureus | |

Test Organism Preparation

Using a stock culture of the test organism, a culture of the test organism was struck onto the culture medium listed above. The bacterial cultures were incubated for 24-48 hours at 35-37° C. (alternate incubation may be utilized for certain strains). To produce a suspension containing approximately 1×10⁸-1×10⁹ CFU/mL, the culture suspension was adjusted with Butterfield's Buffer to yield a suspension matching a 0.5 McFarland turbidity standard.

Preparation of Test Substance

The test substance to be tested was prepared by transferring a 5.95 mL aliquot of the prepared test substance to a sterile vessel for testing procedures. The test substances were assayed for microbial killing activity within 3 hours of preparation.

Test Exposure

A 0.05mL aliquot of the standardized inoculum was added to the test substance representing the start of the test exposure (time=0 hr). The inoculated test substance was immediately mixed thoroughly using a laboratory vortex mixer or other applicable method. The inoculated and mixed test substance was held at the specified temperature.

Subculture

A 1.0 mL aliquot of the inoculated test substance was transferred to 9 mL of neutralizer broth (10× dilution) at each of the times indicated. Four additional 1:10 dilutions in Butterfield's Buffer were also prepared. Using a standard microbiological spread plate count procedure, 1.0 mL aliquots of each dilution (10⁻¹-10⁻⁴) were plated in duplicate to the appropriate recovery media.

A 5.0 mL aliquot of the neutralized sample (10× dilution) was transferred to a sterile 0.2-0.45 μm filter apparatus system pre-wetted with 10 mL sterile diluent. The sample was filter concentrated and the filter was rinsed using ≧50 mL sterile diluent. The filter was then aseptically removed and placed on the surface of the recovery agar medium to determine the percent MRSa survival.

Incubation and Observation

The bacterial subculture plates were incubated for 48±4 hours at 35-37° C. Subculture plates may be refrigerated at 2-8° C. for ≦3 days prior to examination without affecting the results. Following incubation, the subculture tubes and plates were visually examined for growth. The agar plates were enumerated and recorded. Log and percent reductions will be determined for each time point. Representative subcultures demonstrating growth were appropriately examined for confirmation of the viability of the test organism.

Purity Control

A “streak plate for isolation” was performed on the organism culture, and, following incubation, examined in order to confirm the presence of a pure culture. The acceptance criterion for this study control was a pure culture demonstrating colony morphology typical of the test organism.

Neutralizer Sterility Control

A representative sample of neutralizer was incubated and observed. The acceptance criterion for this study control was lack of growth.

Test Population Control

As a control for the viability of the test organism, an equivalent volume of untreated inoculum (0.1 mL) is added to 9.9 mL Butterfield's Buffer (same volume as the test substance). This suspension was neutralized as in the test procedure. The suspension was serially diluted and appropriate dilutions plated using standard microbiological techniques. Following incubation, the organism plates were observed to enumerate the concentration of the test organism present in the test substance at the time of testing (time=0 analysis). The acceptance criteria for this study control is growth and the value is used for calculation purposes.

Initial Suspension Population Control

The prepared test organism suspension will be serially diluted and plated using standard microbiological techniques. Following incubation, the organism plates will be observed to enumerate the concentration of the test organism inoculated into the test substance at the time of testing. The acceptance criteria for this study control is growth at ≧1.0×10⁶ CFU/mL.

Neutralization Controls

To stimulate testing conditions, 9.9 mL of the test substance were inoculated with 0.1 mL Butterfield's Buffer in place of the test organism suspension (NC Suspension).

1. Filtration Neutralization Control:

1.0 mL of the NC Suspension was transferred to 9 mL neutralizing broth and mixed thoroughly. 5.0 mL of the control suspension was filter concentrates and the filter was rinsed as in the test procedure. 1.0 mL of an organism suspension was added containing approximately 100 CFU/mL to the filter apparatus and processed through the apparatus. 1.0 mL of the organism suspension was added to a second filter apparatus to be used as an inoculum and process population control. The filters were aseptically transferred to recovery agar plates and incubated. The acceptance criteria for this study control required that the filtration neutralization control and corresponding population control results be within 1.0 Log of each other.

2. Chemical Neutralization:
1.0 mL of the NC Suspension was transferred to 9 mL of neutralizing broth and mixed thoroughly. 1.0 mL of the neutralized sample was removed and discarded. To the neutralized sample, 1.0 mL of an organism suspension containing approximately 1000 CFU/mL was added and mixed thoroughly. 1.0 mL of neutralized mixture was plated in duplicate to an appropriate recovery medium and incubated. An inoculum population control was performed by adding 1.0 ml of the same organism suspension to 9 mL of Butterfield's Buffer and plating in duplicate prior to incubation. The acceptance criteria for this study control required the chemical neutralization control and corresponding population control results to be within 1.0 Log of each other.
   Test Substance Performance Criteria

This study is designed to examine the rate-of-kill of the test substance after inoculation with a test organism. Results were expressed in percent and log reduction of the test organism. Minimum percent and log reduction values do not exist to specify a “passing” or “failing” test substance.

Control Acceptance Criteria

The study controls must perform according to the criteria detailed in the study controls description section.

Data Analysis

The number of colony forming units (CFU), the percent reduction in MRSa count and the log reduction were calculated as follows.

Test Data CFU/mL:
(avg. # colonies found/plate @ dilution used) (dilution factor) (volume of neutralized solution)/(volume plated)
Percent Reduction: [1−(test survivors/test population control)]×100
Log₁₀ Reduction: Log₁₀ (test population control)—Log₁₀ (test survivors)
Further details of the microbial time-killing studies can be found in:
1. American Society for Testing and Materials (ASTM). E2315-03, Guide for Assessment of Microbiocidal Activity Using a Time-Kill Procedure, Volume 11.05, Copyright 2005 ASTM International; and
2. Food and Drug Administration. Tentative Final Monograph for Healthcare Antiseptic Drug Products; Proposed rule. Code of Federal Regulations, 21 CFR parts 333 and 369. Jun. 17, 1994.
   Results

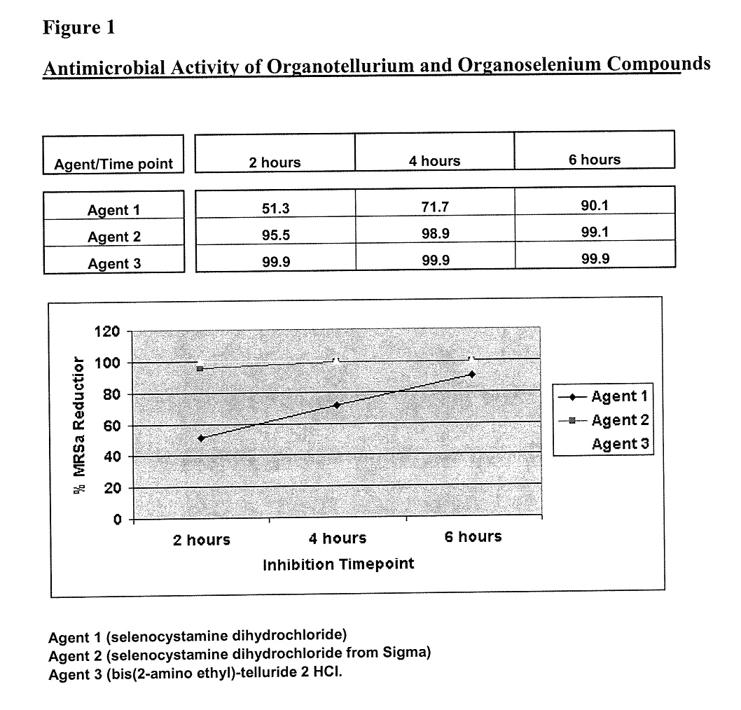

The MRSa culture tested had a bacterial count of 1.13×10⁶ CFU/mL in the test solution/organism mixture. The results, shown in FIG. 1, demonstrate that Agent 1 (selenocystamine dihydrochloride) effected a 51.3% reduction (0.31 log₁₀ reduction) of MRSa following a 2 hour exposure, a 71.7% reduction (0.54 log₁₀ reduction) of MRSa following a 4 hour exposure, and a 90.1% reduction (1.004 log₁₀ reduction) of MRSa following a 6 hour exposure at room temperature (20° C.). In contrast, commercially obtained Agent 2 (selenocystamine dihydrochloride from Sigma 100 mg) demonstrated a 95.5% reduction (1.34 log₁₀ reduction) of MRSa following a 2 hour exposure, a 98.9% reduction (1.946 log₁₀ reduction) of MRSa following a 4 hour exposure, and a 99.1% reduction (2.024 log₁₀ reduction) of MRSa following a 6 hour exposure at room temperature (20° C.). Accordingly, the commercially obtained selenocystamine dihydrochloride appeared more microbially active. Surprisingly, Agent 3 (bis(2-amino ethyl)-telluride 2 HCl) demonstrated a >99.9% reduction (3.4 log₁₀ reduction) of MRSa following a 2 hour exposure, and a >99.9% reduction (>4.1 log₁₀ reduction) of MRSa following a 4 and 6 hour exposures with at room temperature (20° C.). Therefore the organotellurium compound tested was highly active as an antimicrobial.

Further testing of the Seleno methacrylate, 2-selenocyanatoethyl methacrylate, demonstrates that it provides a >99.9% reduction (>4.1 log₁₀ reduction) of MRSa following 2, 4 and 6 hour exposures at room temperature (20° C.).

The results demonstrate that organotellurium compounds, as well as organoselenium compounds, possess a surprisingly potent antimicrobial killing activity against human pathogenic bacteria.

### Example 13

### Antimicrobial Activity of Organotellurium and Organoselenium Compounds

To demonstrate that the potent antimicrobial effects are not specific to selenocystamine dihydrochloride and bis (2-amino)-telluride 2HCl, but are a feature of organotellurium and organoselenium compounds in general, additional organic and inorganic selenium and tellurium compositions were tested as described above.

Test Substance 1 was 200 mg K₂TeO₃ (Potassium tellurite, an inorganic tellurium salt), Test Substance 2 was 100 mg DPDT (Diphenyl ditelluride), and Test Substance Sample 3: 100 mg TMS (Phenylselenomethyl)trimethylsilane.

Starting with ˜3-4×10⁶ CFU/mL Staphylococcus aureus-MRSA (ATCC 33592), the number of survivors following exposure times of 2, 4 and 6 hours was determined using the procedures described above. The results showed that Test Substance 1 (the inorganic tellurium salt) effected very little to no reduction of MRSA at all time points. In contrast, Test Substance 2, an aromatic organotellurium compound (diphenyl ditelluride from Sigma Aldrich), effected a significant reduction (4−5+log) of MRSA at all time points tested. Similarly, Test Substance 3, an aromatic and alophatic organoselenium compound ((phenylelenomethyl)trimethylsilane), effected a significant reduction (4−5+log) of MRSA at all time points. Their potent antimicrobial activity is a feature of both organotellurium and organoselenium compounds.

### Example 14

### Preparing Antimicrobial Organotellurium and Organoselenium Articles

In this example, organotellurium-permeated and organoselenium-permeated articles are prepared. The articles are fabricated to afford 10 mg/cm², 50 mg/cm², 100 mg/cm², 250 mg/cm², 500 mg/cm², or 1000 mg/cm² of Se or Te at the surface of the article. Based upon the % Se or % Te in the compound and the drying profile of the articles, one can reproducibly achieve these target concentrations at the surface by admixing the appropriate amount of compound into the coating which is applied to a substrate and then drying at the prerequisite temperature and dwell time. As a rule, the more open time you afford the article, the more migration to the surface will be achieved during drying (see Example 8, above). In general, open time is dependent upon the temperature and the dwell time and specifically, longer dwell times at lower temperatures afford longer open times.

In the case of water-based coatings, the ability of molecules to migrate to the surface is almost exclusively dependent upon the open time. Other factors that may also influence migration are molecular size and hydrophilicity. In particular, smaller molecules will migrate faster and better than larger molecules and the hydrophilicity of the molecule will also have some influence on surface migration particularly during the drying phase because the more water soluble or hydrophilic the compound is, the more readily it will follow the water out of the coating and concentrate at the surface. The key to antimicrobial activity is found in the structure of the parent compound such that it is able to react with sulfhydryl groups and oxygen to produce germicidal superoxide. The key to surface migration is the ability of the small, hydrophilic molecule to stick with the water, through the coating matrix and concentrate at the surface of the coating. Therefore any of the bis(aminoethyl)chalcogenides or dichalcogenides mentioned above have been shown to be active against MRSa and can be isolated as their inorganic salts (HCl, HBr, HF or HI) or salts of organic acids such as formic, acetic, pyruvic, lactic, oxalic, succinic, maleic, methylsulfonic, or toluenesulfonic salts or acids.

The open time of the solvent based coatings, which is also dictated by temperature and dwell time, is critical to the successful migration of the organo chalcogenides and dichalcogenides to the surface. The open time of the solvent based coating can further be extended by the addition of a slowly evaporating co-solvent into the formulation thus allowing the bioactive material to “blush” to the surface more easily. Matching the hydrophobicity of the compound to the solvent and/or co-solvent will also contribute to the enhancing both the rate of migration and the concentration of the compound at the surface.

The amount of compound that has migrated to the surface is then quantitatively determined by Chemiluminescence or X-Ray Fluorescence (e.g., as described in Examples 6 and 7, above).

### Example 15

### Antimicrobial Activity of Organotellurium and Organoselenium Articles

In this example, the antimicrobial activity of an organotellurium-coated article and an organoselenium-coated article is detected and measured.

A film of MRSa bacterial cells dried on a surface of glass carriers is treated using a 1 in² section of either the control tape or the test tape. The tape is then applied over the inoculated area of the slide and is firmly pressed in place. Any resultant air bubbles are smoothed out to ensure even contact at the tape/slide interface. The carriers are held at room temperature for four hours. Following the 4-hour exposure, each tape is carefully removed from its carrier. The tapes and their respective treated carriers are separately sub-cultured by adding them to individual jars of neutralizing subculture media. The jars containing the tape are mixed under vortex for one minute and are then sonicated for ten minutes. The jars containing the glass carrier slides are swirled by hand to mix. Ten-fold serial dilutions are prepared from each jar and then 1.0 mL of each of the 10⁰-10⁻³ dilutions are individually spread plated in duplicate onto BAP (Tryptic Soy Agar with 5% Sheep Blood). The subculture plates are incubated for 48+4 hours at 35-37° C. prior to examination. Representative plates showing growth are subcultured, stained, and biochemically assayed to confirm the presence of the MRSa test organism. Appropriate controls including purity, carrier sterility, neutralizing subculture medium sterility, viability, neutralization confirmation, carrier population, background, and antimicrobial resistance (Kirby-Bauer) controls are run to support the accuracy and validity of the test.

Equivalents

Those skilled in the art will recognize, or be able to ascertain, using no more than routine experimentation, numerous equivalents to the specific embodiments described specifically herein. Such equivalents are intended to be encompassed in the scope of the following claims.

We claim:

1. An antimicrobial article comprising a substrate and an organotellurium compound, wherein the organotellurium compound is present in an amount sufficient to treat or prevent the growth or spread of an infectious agent through cutaneous contact of the antimicrobial article with a subject.

2. The antimicrobial article of claim 1, wherein the organotellurium compound has the structure:
R—Te,
where Te is tellurium and R is any organic chemical group.

3. The antimicrobial article of claim 1, wherein the organotellurium compound has the structure:
R—Te—Te—R′,
where Te are tellurium atoms, and R and R′ are each, independently, any organic chemical group.

4. The antimicrobial article of claim 2, wherein the organotellurium compound includes an R group that is a substituted or unsubstituted, saturated or unsaturated, alkyl group having from 1 to about 12 carbon atoms.

5. The antimicrobial article of claim 4, wherein the alkyl group is substituted with one or more substituents selected from the group consisting of methyl, amino, halo(chloro), nitro, methoxy, hydroxy, carboxylate, vinyl, allyl, alkylsilane and combinations thereof.

6. The antimicrobial article of claim 2, wherein the organotellurium compound includes an R group that is a substituted or unsubstituted, saturated or unsaturated, aryl group.

7. The antimicrobial article of claim 6, wherein the aryl group is selected from the group consisting of: phenyl, pyridinium, imidazole, oxazine, and naphthyl.

8. The antimicrobial article of claim 6, wherein the aryl group is substituted with one or more substituents selected from the group consisting of methyl, amino, halo(chloro), nitro, methoxy, hydroxy, carboxylate, vinyl, allyl, alkylsilane and combinations thereof.

9. The antimicrobial article of claim 1, wherein the organotellurium compound is an organotellurium carboxylic acid compound, or alkyl ester thereof.

10. The antimicrobial article of claim 9, wherein the organotellurium carboxylic acid compound, or alkyl ester thereof, is selected from the group consisting of tellurocyanatoacetic acid, 3-tellurocyanatopropionic acid, 2-tellurocyanotopropionic acid, ditellurocyanatodiacetic acid, ditellurocyanatodipropionic acid, and mixtures thereof.

11. The antimicrobial article of claim 1, wherein the organotellurium compound is selected from the group consisting of tellurocyanatoethyl amine, 3-tellurocyanatopropyl amine, tellurocystamine dihydrochloride and 2-telluroethyl methacrylate.

12. The antimicrobial article of claim 1, wherein the organotellurium compound is selected from the group consisting of tellurodicarboxylic acid, organotellurium diamines, organotellurium monoamines (and amides thereof), organotellurium amines (e.g., cystamine), telluroamino acids (e.g., L-tellurocystine), organotellurium carboxylic acids and amides thereof), telluroacrylates and esters and amides thereof, telluromethacrylates and esters and amides thereof, tellurourethanes, telluroureas, and tellurofatty acids and esters and amides thereof), tellurochloroprene, tellurobromoprene, tellurostyrene, tellurobutadiene, telluroacrylonitrile, and mixtures thereof.

13. The antimicrobial article of claim 1, wherein the organotellurium compound is a telluro adduct of oleic acid, or an alkyl ester thereof.

14. The antimicrobial article of claim 1, wherein the organotellurium compound is a polymer, or mixture of polymers, selected from the group consisting of telluroamino acids, 2-substituted organotellurium oxazolines, 2-substituted organotellurium oxazines, telluroacrylate, telluromethacrylate, tellurourethane, tellurourea, tellurochloroprene, tellurobromoprene, tellurostyrene, tellurobutadiene, and telluroacrylonitrile.

15. The antimicrobial article of claim 1, wherein the organotellurium compound is selected from the group 2,2′-tellurodiethanamine dihydrochloride, and 2,2′-ditellurodiethanamine dihydrochloride.

16. The antimicrobial article of claim 1, wherein the organotellurium compound is present at a concentration of at least about 3 μg/cm² of Te (about 23.5 nmole/cm² Te).

17. The antimicrobial article of claim 1, wherein the organotellurium compound is present at no more than about 200 μg/cm² of Te (about 1.57 μmole/cm² Te).

18. The antimicrobial article of claim 1, wherein the organotellurium compound is present at about 3 μg/cm² of Te (about 23.5 nmole/cm² Te) to about 100 μg/cm² of Te (about 784 nmole/cm² Te).

19. The antimicrobial article of claim 18, wherein the organotellurium compound is present at about 10 μg/cm² of Te (about 78.4 nmole/cm² Te) to about 20 μg/cm² of Te (about 157 nmole/cm² Te).

20. The antimicrobial article of claim 1, wherein the article comprises at least one surface having about 3 ug of elemental tellurium per square centimeter of surface area.

21. The antimicrobial article of claim 1, wherein the organotellurium compound is non-covalently associated with the article.

22. The antimicrobial article of claim 21, wherein the organotellurium compound is formulated in a formulation that is applied to at least one surface of the antimicrobial article.

23. The antimicrobial article of claim 22, wherein the formulation comprising the organotellurium is a pressure-sensitive adhesive.

24. The antimicrobial article of claim 22, wherein the formulation comprising the organotellurium is a cohesive agent.

25. The antimicrobial article of claim 22, wherein at least 50% of the organotellurium is bioavailable at the surface of the article.

26. The antimicrobial article of claim 22, wherein at least 70% of the organotellurium is bioavailable at the surface of the article.

27. The antimicrobial article of claim 1, wherein the article is a tape.

28. The antimicrobial article of claim 1, wherein the article is a bandage.

29. A method of treating or preventing an infectious disease spread through cutaneous contact of a subject with a medical or sports article comprising:
contacting the subject topically with a medical or sports article having, on at least one surface, an organic tellurium compound, or formulation thereof, capable of generating superoxide radicals in the presence of an infectious agent;
wherein the superoxide radicals generated by the organic tellurium compound inhibit or inactivate an agent of the infectious disease and thereby treat or prevent the infectious disease in the subject.

30. A method of treating or preventing the development or transmission of an infectious disease in a subject through the use of an adhesive or cohesive article applied cutaneously to the subject comprising:
providing an adhesive or cohesive article having, on at least one surface, an organic tellurium compound, or formulation thereof, capable of generating superoxide radicals in the presence of an infectious agent at a surface of the article; and
applying the surface of the adhesive or cohesive article to the subject,
wherein the superoxide radicals generated by the organic tellurium compound at the applied surface of the article inhibit or inactivate the agent of the infectious disease and thereby treat or prevent the infectious disease in the subject.

31. A method of making an antimicrobial adhesive or cohesive formulation comprising:
providing an adhesive or cohesive formulation; and
adding to the adhesive or cohesive formulation an organic tellurium compound, or formulation thereof, capable of generating superoxide radicals in the presence of an infectious agent,
thereby making the antimicrobial adhesive or cohesive formulation.

32. A method of making an antimicrobial adhesive or cohesive article comprising:
providing an adhesive or cohesive formulation;
adding to the adhesive or cohesive formulation an organic tellurium compound, or formulation thereof, capable of generating superoxide radicals in the presence of an infectious agent to produce an antimicrobial adhesive or cohesive formulation; and
applying the antimicrobial adhesive or cohesive formulation to at least one surface of the article,
thereby making the antimicrobial adhesive or cohesive article.

33. The method of any of claims 29-32, wherein the agent of infectious disease is a bacteria.

34. The method of claim 33, wherein the bacteria is Staphylococcus aureus.

35. The method of claim 33, wherein the bacteria is Pseudomonas aeruginosa.

36. The method of claim 33, wherein the bacteria is Acinetobacter baumanii.

37. The method of any of claims 29-32, wherein the agent of infectious disease is a fungus.

38. The method of any of claims 29-32, wherein the agent of infectious disease is a protozoa.

39. The method of any of claims 29-32, wherein the agent of infectious disease is a virus.

40. The method of any of claims 29-32, wherein the organic tellurium compound, or formulation thereof, does not comprise a thiol group or a thiol-containing compound.

41. The method of claim 40, wherein the organic tellurium compound, or formulation thereof, does not comprise glutathione.

42. The method of any of claims 29-32, wherein the subject is a mammal or a marsupial.

43. The method of claim 42, wherein the mammal is selected from the group consisting of: a mouse, a rat, a dog, a cat, a cow, a horse, a goat and a pig.

44. The method of claim 42, wherein the subject is a human.

45. The method of any of claims 29-32, wherein the organic tellurium compound is selected from the group consisting of tellurocystine and telluromethionine.

46. The method of any of claims 29-32, wherein the organotellurium compound is a carboxylic acid compound, or alkyl ester thereof, selected from the group consisting of tellurocyanatoacetic acid, 3-tellurocyanatopropionic acid, 2-tellurocyanatopropionic acid, ditellurocyanatodiacetic acid, ditellurocyanatodipropionic acid, and mixtures thereof.

47. The method of any of claims 29-32, wherein the organotellurium compound is selected from the group consisting of tellurocyanatoethyl amine, 3-tellurocyanatopropyl amine, tellurocystamine dihydrochloride and 2-tellurocyanatoethyl methacrylate.

48. The method of any of claims 29-32, wherein the organotellurium compound is selected from the group consisting of: tellurodicarboxylic acid, organotellurium diamines, organotellurium monoamines (and amides thereof), organotellurium amines (e.g., cystamine), telluroamino acids (e.g., L-tellurocystine), organotellurium carboxylic acids (and amides thereof), telluroacrylates (and esters and amides thereof), telluromethacrylates (and esters and amides thereof), tellurourethanes, telluroureas, and tellurofatty acids (and esters and amides thereof), tellurochloroprene, tellurobromoprene, tellurostyrene, tellurobutadiene, telluroacrylonitrile, and mixtures thereof.

49. The method of any of claims 29-32, wherein the organotellurium compound is a telluro adduct of oleic acid, or an alkyl ester thereof.

50. The method of any of claims 29-32, wherein the organotellurium compound is a polymer, or mixture of polymers, of an organotellurium compound selected from the group consisting of telluroamino acids, 2-substituted organotellurium oxazolines, 2-substituted organotellurium oxazines, telluroacrylate, telluromethacrylate, tellurourethane, tellurourea, tellurochloroprene, tellurobromoprene, tellurostyrene, tellurobutadiene, and telluroacrylonitrile.

51. The method of any of claims 29, 30, or 32, wherein the organic tellurium compound, or formulation thereof, is non-covalently associated with the article.

52. The method of any of claims 29, 30, or 32, wherein the organic tellurium compound, or formulation thereof, is covalently associated with the article.

53. The method of any of claims 29, 30, or 32, wherein the article is an adhesive tape or bandage.

54. The method of any of claims 29, 30, or 32, wherein the article is a cohesive tape or bandage

55. The method of claim 29 or 30, wherein the surface of the article having the organic tellurium compound, or formulation thereof, further comprises a pressure-sensitive adhesive.

56. The method of claim 31 or 32, wherein the adhesive or cohesive formulation is a pressure-sensitive adhesive.

57. The method of claim 56, wherein the pressure-sensitive adhesive is an acrylic water-based or solvent-based pressure-sensitive adhesive.

58. The method of claim 56, wherein the pressure-sensitive adhesive is a hot-melt adhesive.

59. The method of claim 32, wherein at least about half of the elemental tellurium from the organic tellurium compound, or formulation thereof, exists in an active state that is capable of generating superoxide radicals on the surface of the antimicrobial adhesive or cohesive article.

60. The method of any of claims 29, 30, or 32, wherein the article has at least one surface having at least about 2 ug of elemental tellurium per square centimeter of surface area.

61. The method of claim 60, wherein the article has at least one surface having at least about 6 ug of elemental tellurium per square centimeter of surface area.

62. An antimicrobial article having, on at least one surface, an effective amount of an organic tellurium compound, or formulation thereof, capable of generating superoxide radicals in the presence of an infectious agent compound, wherein the organic tellurium compound is non-covalently associated with the article and an effective amount of the organic tellurium compound, or formulation thereof, is retained on the surface of the article when the article is in cutaneous contact with a subject.

63. An antimicrobial adhesive or cohesive article having, on at least one surface, an antimicrobial cohesive or adhesive formulation comprising an effective amount of an organic tellurium compound, or formulation thereof, capable of generating superoxide radicals in the presence of an infectious agent or reduced thiol compound, wherein the organic tellurium compound is non-covalently associated with the article and an effective amount of the organic tellurium compound, or formulation thereof, is retained on the surface of the article when the article is in cutaneous contact with a subject.

64. An antimicrobial adhesive or cohesive article product of the process comprising:
providing an adhesive or cohesive formulation;
adding to the adhesive or cohesive formulation an organic tellurium compound, or formulation thereof, capable of generating superoxide radicals in the presence of an infectious agent to produce an antimicrobial adhesive or cohesive formulation; and
applying the antimicrobial adhesive or cohesive formulation to at least one surface of the article to produce the antimicrobial adhesive or cohesive article,
wherein the resulting antimicrobial adhesive or cohesive article carries, on at least one surface, an effective amount of the organic tellurium compound, or formulation thereof, that is capable of generating superoxide radicals in the presence of an infectious agent.

65. An antimicrobial adhesive or cohesive article having, on at least one surface, an antimicrobial cohesive or adhesive formulation comprising an effective amount of an organic tellurium compound, or formulation thereof, capable of generating superoxide radicals in the presence of an infectious agent or a reduced thiol compound, wherein the organic tellurium compound is covalently associated with the article and an effective amount of the organic tellurium compound, or formulation thereof, is retained on the surface of the article when the article is in cutaneous contact with a subject.

66. The antimicrobial article of any of claims 62-65, wherein the effective amount of the organic tellurium compound, or formulation thereof, that is retained on the surface of the article when the article is in cutaneous contact with the subject is sufficient to inhibit or inactivate an agent of infectious disease.

67. The antimicrobial article of claim 66, wherein the agent of infectious disease is a bacteria.

68. The antimicrobial article of claim 67, wherein the bacteria is Staphylococcus aureus.

69. The antimicrobial article of claim 67, wherein the bacteria is Pseudomonas aeruginosa or Acinetobacter baumannii.

70. The antimicrobial article of claim 66, wherein the agent of infectious disease is a fungus.

71. The antimicrobial article of claim 66, wherein the agent of infectious disease is a protozoa.

72. The antimicrobial article of claim 66, wherein the agent of infectious disease is a virus.

73. The antimicrobial article of any of claims 62-65, wherein the antimicrobial article having the organic tellurium compound, or formulation thereof, does not further have a thiol group or a thiol-containing compound.

74. The antimicrobial article of claim 73, wherein the antimicrobial article does not further comprise glutathione.

75. The antimicrobial article of claim 62, 63 or 65, wherein the subject is a mammal.

76. The antimicrobial article of claim 75, wherein the subject is a human.

77. The antimicrobial article of claims 62-65, wherein the organic tellurium compound is selected from the group consisting of tellurocystine and telluromethionine.

78. The antimicrobial article of claims 62-65, wherein the organic tellurium compound is an organotellurium carboxylic acid compound, or alkyl ester thereof, selected from the group consisting of tellurocyanatoacetic acid, 3-tellurocyanatopropionic acid, 2-tellurocyanatopropionic acid, ditellurocyanatodiacetic acid, ditellurocyanatodipropionic acid, and mixtures thereof.

79. The antimicrobial article of claims 62-65, wherein the organic tellurium compound is an organotellurium compound selected from the group consisting of tellurocyanatoethyl amine, 3-tellurocyanatopropyl amine, tellurocystamine dihydrochloride and 2-tellurocyanatoethyl methacrylate.

80. The antimicrobial article of claims 62-65, wherein the organic tellurium compound is a telluro adduct of oleic acid, or an alkyl ester thereof.

81. The antimicrobial article of claims 62-65, wherein the article is an adhesive tape or bandage.

82. The antimicrobial article of claims 62-65, wherein the article is a cohesive tape or bandage

83. The antimicrobial article of claims 62-65, wherein the surface of the article having the organic tellurium compound, or formulation thereof, further carries a pressure-sensitive adhesive.

84. The antimicrobial article of claim 83, wherein the pressure-sensitive adhesive is an acrylic water-based or solvent-based pressure-sensitive adhesive.

85. The antimicrobial article of claim 83, wherein the pressure-sensitive adhesive is a hot-melt adhesive.

86. The antimicrobial article of claims 62-65, wherein at least about half of the elemental tellurium from the organic tellurium compound, or formulation thereof, exists in an active state that is capable of generating superoxide radicals on the surface of the antimicrobial article.

87. The antimicrobial article of claims 62-65, wherein the article has at least one surface having at least about 2 ug of elemental tellurium per square centimeter of surface area.

88. The antimicrobial article of claim 87, wherein the article has at least one surface having about 6 ug of elemental tellurium per square centimeter of surface area.

89. An antimicrobial article comprising a substrate and an inorganic or organic selenium compound, wherein the inorganic or organic selenium compound is present in an amount sufficient to treat or prevent the growth or spread of an infectious agent through cutaneous contact of the antimicrobial article with a subject.

90. The antimicrobial article of claim 89, wherein the organoselenium compound has the structure:
R—Se,
where Se is selenium and R is any organic chemical group.

91. The antimicrobial article of claim 89, wherein the organoselenium compound has the structure:
R—Se—Se—R′,
where Se are selenium atoms, and R and R′ are each, independently, any organic chemical group.

92. The antimicrobial article of claim 90, wherein the organoselenium compound includes an R group that is a substituted or unsubstituted, saturated or unsaturated, alkyl group having from 1 to about 12 carbon atoms.

93. The antimicrobial article of claim 92, wherein the alkyl group is substituted with one or more substituents selected from the group consisting of methyl, amino, halo(chloro), nitro, methoxy, hydroxy, carboxylate, vinyl, allyl, alkylsilane and combinations thereof.

94. The antimicrobial article of claim 90, wherein the organoselenium compound includes an R group that is a substituted or unsubstituted, saturated or unsaturated, aryl group.

95. The antimicrobial article of claim 94, wherein the aryl group is selected from the group consisting of: phenyl, pyridinium, imidazole, oxazine, and naphthyl.

96. The antimicrobial article of claim 89, wherein the aryl group is substituted with one or more substituents selected from the group consisting of methyl, amino, halo(chloro), nitro, methoxy, hydroxy, carboxylate, vinyl, allyl, alkylsilane and combinations thereof.

97. The antimicrobial article of claim 89, wherein the organoselenium compound is an organoselenium carboxylic acid compound, or alkyl ester thereof.

98. The antimicrobial article of claim 97, wherein the organoselenium carboxylic acid compound, or alkyl ester thereof, is selected from the group consisting of selenocyanatoacetic acid, 3-selenocyanatopropionic acid, 2-selenocyanotopropionic acid, diselenocyanatodiacetic acid, diselenocyanatodipropionic acid, and mixtures thereof.

99. The antimicrobial article of claim 89, wherein the organoselenium compound is selected from the group consisting of selenocyanatoethyl amine, 3-selenocyanatopropyl amine, selenocystamine dihydrochloride and 2-selenocyanatoethyl methacrylate.

100. The antimicrobial article of claim 89, wherein the organoselenium compound is selected from the group consisting of selenodicarboxylic acid, organoselenium diamines, organoselenium monoamines (and amides thereof), organoselenium amines (e.g., cystamine), selenoamino acids (e.g., L-selenocystine), organoselenium carboxylic acids and amides thereof), selenoacrylates and esters and amides thereof, selenomethacrylates and esters and amides thereof, selenourethanes, selenoureas, and selenofatty acids and esters and amides thereof), selenochloroprene, selenobromoprene, selenostyrene, selenobutadiene, selenoacrylonitrile, and mixtures thereof.

101. The antimicrobial article of claim 89, wherein the organoselenium compound is a seleno adduct of oleic acid, or an alkyl ester thereof.

102. The antimicrobial article of claim 89, wherein the organoselenium compound is a polymer, or mixture of polymers, selected from the group consisting of selenoamino acids, 2-substituted organoselenium oxazolines, 2-substituted organoselenium oxazines, selenoacrylate, selenomethacrylate, selenourethane, selenourea, selenochloroprene, selenobromoprene, selenostyrene, selenobutadiene, and selenoacrylonitrile.

103. The antimicrobial article of claim 89, wherein the organoselenium compound is selected from the group 2,2′-selenodiethanamine dihydrochloride, and 2,2′-diselenodiethanamine dihydrochloride.

104. The antimicrobial article of claim 89, wherein the organoselenium compound is present at a concentration of at least about 3 {μg/cm² of Se (about 38.0 nmole/cm² Se).

105. The antimicrobial article of claim 89, wherein the organoselenium compound is present at no more than about 200 μg/cm of Se (about 2.54 μmole/cm² Se).

106. The antimicrobial article of claim 89, wherein the organoselenium compound is present at about 3 μg/cm² of Se (about 38.0 nmole/cm² Se) to about 100 μg/cm² of Se (about 1,266 nmole/cm² Se).

107. The antimicrobial article of claim 106, wherein the organoselenium compound is present at about 10 μg/cm² of Se (about 126.6 nmole/cm² Se) to about 20 μg/cm² of Se (about 253 nmole/cm² Se).

108. The antimicrobial article of claim 89, wherein the article comprises at least one surface having about 3 ug of elemental selenium per square centimeter of surface area.

109. The antimicrobial article of claim 89, wherein the organoselenium compound is non-covalently associated with the article.

110. The antimicrobial article of claim 109, wherein the organoselenium compound is formulated in a formulation that is applied to at least one surface of the antimicrobial article.

111. The antimicrobial article of claim 110, wherein the formulation comprising the organoselenium is a pressure-sensitive adhesive.

112. The antimicrobial article of claim 110, wherein the formulation comprising the organoselenium is a cohesive agent.

113. The antimicrobial article of claim 110, wherein at least 50% of the organoselenium is bioavailable at the surface of the article.

114. The antimicrobial article of claim 110, wherein at least 70% of the organoselenium is bioavailable at the surface of the article.

115. The antimicrobial article of claim 89, wherein the article is a tape.

116. The antimicrobial article of claim 89, wherein the article is a bandage.

117. The antimicrobial article of claim 89, wherein the inorganic selenium compound is a salt of selenite (SeO₃⁻²).

118. The antimicrobial article of claim 117, wherein the selenite is sodium selenite (Na₂SeO₃).
